(19) United States
(12) Patent Application Publication
(10) Pub. No.: US 2020/0187864 A1
(43) Pub. Date: Jun. 18, 2020

(54) MODIFICATION OF HEART FAILURE MONITORING ALGORITHM TO ADDRESS FALSE DETERMINATIONS

(71) Applicant: Medtronic, Inc., Minneapolis, MN (US)

(72) Inventor: Vinod Sharma, Maple Grove, MN (US)

(21) Appl. No.: 16/222,088

(22) Filed: Dec. 17, 2018

(51) Int. Cl. A61B 5/00

(52) U.S. Cl. CPC A61B 5/7275; A61B 5/7221

(57) ABSTRACT

In some examples, a system comprises one or more medical devices configured to, for each of a plurality of periods, determine a respective value for each of a plurality of parameters of a patient and processing circuitry. The processing circuitry can for each of the plurality of periods, execute an algorithm to determine at least one of a heart failure risk score or status for the patient based on the determined values for the period, determine a number of the determined heart failure risk scores or statuses that were false determinations, determine that the number of false determinations for the patient satisfies a false determination threshold, and modify the algorithm for the patient in response to the determination that the number of false determinations for the patient satisfies the false determination threshold.

### TECHNICAL FIELD

The disclosure relates to patient health, and more particularly to medical devices and techniques that monitor cardiac health of a patient.

### BACKGROUND

Various types of implantable and/or external medical devices may collect and store data. These devices include microprocessors for processing collected data for various purposes. The devices may process the collected data to detect and classify certain patient conditions. The patient may benefit from the delivery of therapy, and the processed data may guide treatment of the patient to address one or more of the patient conditions.

Chronic heart failure (HF), for example, can occur when a heart is unable to consistently pump blood at an adequate rate in response to the filling pressure. Global health care systems incur billions of dollars each year due to heart failure hospitalizations (HFHs). Identifying patients at risk of HFH to enable timely intervention and prevent expensive hospitalization remains a challenge. Medical devices may be configured to acquire data for a variety of diagnostic metrics that change with HF status and collectively have the potential to signal an increasing risk of HFH. Such a diagnostic medical device may, in some examples, also provide therapy to treat HF, such as cardiac resynchronization therapy (CRT).

### SUMMARY

In general, this disclosure is directed to techniques for implementing an algorithm to determine a HF score and/or status based on a plurality of diagnostic metrics. More particularly, this disclosure is directed to techniques for modifying the algorithm based on false determinations of the HF score and/or status by the algorithm. The false determinations may be false positive determinations and/or false negative determinations. Modification of the algorithm according to the techniques described herein may enhance sensitivity and specificity of the algorithm in detecting patients at risk of HF, e.g., an HFH event and improve overall performance of the algorithm including false alert rate.

In one example, a system comprises one or more medical devices configured to, for each of a plurality of periods, determine a respective value for each of a plurality of parameters of a patient, and processing circuitry. The processing circuitry is configured to, for each of the plurality of periods, determine at least one of a heart failure risk score or status for the patient based on the determined values for the period using one or more thresholds, determine a number of the determined heart failure risk scores or statuses that were false determinations, determine that the number of false determinations for the patient satisfies a false determination criterion, and modify at least one of the one or more thresholds for the patient in response to the determination that the number of false determinations for the patient satisfies the false determination criterion.

In another example, a system comprises one or more medical devices configured to, for each of a plurality of periods, determine a respective value for each of a plurality of parameters of a patient, and processing circuitry. The processing circuitry is configured to, for each of the plurality of periods, determine at least one of a heart failure risk score or status for the patient based on the determined values for the period using one or more thresholds, determine a number of the determined heart failure risk scores or statuses that were false determinations, determine that the number of false determinations for the patient satisfies a false determination criterion, and automatically modify at least one of the one or more thresholds for the patient in response to the determination that the number of false determinations for the patient satisfies the false determination criterion.

In another example, a method comprises for each of a plurality of periods, determining a respective value for each of a plurality of parameters of a patient with one or more medical devices, for each of the plurality of periods, determining at least one of a heart failure risk score or status for the patient based on the determined values for the period using one or more thresholds, determining a number of the determined heart failure risk scores or statuses for the patient that were false determinations, determining that that the number of false determinations for the patient satisfies a false determination criterion, and modifying at least one of the one or more thresholds for the patient in response to the determination that the number of false determinations for the patient satisfies the false determination criterion.

In another example, a computer-readable storage medium comprises instructions that, when executed by processing circuitry, cause the processing circuitry to, for each of a plurality of periods, determine a respective value for each of a plurality of parameters of a patient with one or more medical devices, for each of the plurality of periods, determine at least one of a heart failure risk score or status for the patient using one or more thresholds, determine a number of the determinations for the patient that were false, determine that that the number of false determinations for the patient satisfies a false determination criterion, and modify at least one of the one or more thresholds for the patient in response to the determination that the number of false determinations for the patient satisfies the false determination criterion.

In another example, a system comprises one or more medical devices configured to, for each of a plurality of periods, determine a respective value for each of a plurality of parameters of a patient, and processing circuitry. The processing circuitry is configured to, for each of the plurality of periods, execute an algorithm to determine at least one of a heart failure risk score or status for the patient based on the determined values for the period, determine a number of the determined heart failure risk scores or statuses that were false determinations, determine that the number of false determinations for the patient satisfies a false determination threshold, and modify the algorithm for the patient in response to the determination that the number of false determinations for the patient satisfies the false determination threshold.

In another example, a method comprises, for each of a plurality of periods, determining a respective value for each of a plurality of parameters of a patient with one or more medical devices, for each of the plurality of periods, executing an algorithm to determine at least one of a heart failure risk score or status for the patient based on the determined values for the period, determining a number of the determined heart failure risk scores or statuses that were false determinations, determining that the number of false determinations for the patient satisfies a false determination threshold, and modifying the algorithm for the patient in response to the determination that the number of false determinations for the patient satisfies the false determination threshold.

This summary is intended to provide an overview of the subject matter described in this disclosure. It is not intended to provide an exclusive or exhaustive explanation of the methods and systems described in detail within the accompanying drawings and description below. The details of one or more aspects of the disclosure are set forth in the accompanying drawings and the description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual drawing illustrating an example system configured to transmit diagnostic information indicative of heart failure, the system including an implantable medical device (IMD) coupled to implantable medical leads, in accordance with one or more aspects of this disclosure.

FIG. 2A is a conceptual drawing illustrating the example IMD and leads of FIG. 1 in conjunction with a heart, in accordance with one or more aspects of this disclosure.

FIG. 2B is a conceptual drawing illustrating the example IMD of FIG. 1 coupled to a different configuration of implantable medical leads in conjunction with a heart, in accordance with one or more aspects of this disclosure.

FIG. 3 is a functional block diagram illustrating an example configuration of the IMD of FIG. 1.

FIG. 4 is a block diagram illustrating an example system that includes an external device, such as a server, and one or more computing devices that are coupled to the IMD and external device shown in FIG. 1 via a network.

FIG. 5 is a functional block diagram illustrating an example configuration of an external device, such as a server, configured to run an algorithm and collect diagnostic data.

FIG. 6 is a flow diagram of an example technique for implementing an algorithm to determine a HF score and/or status based on patient diagnostic data.

FIG. 7 is a flow diagram of an example technique for monitoring and adjusting an algorithm for determining a HF score and/or status.

FIG. 8 is a flow diagram of an example technique for monitoring and adjusting an algorithm for determining a HF score and/or status based on identification of negative and positive false determinations.

FIG. 9 is a flow diagram of an example technique for monitoring and adjusting an algorithm for determining a HF score and/or status in response to false positives.

FIG. 10 is a flow diagram of an example technique for monitoring and adjusting an algorithm for determining a HF score and/or status in response to false negatives.

### DETAILED DESCRIPTION

In general, this disclosure describes example techniques, systems, and devices, for executing an algorithm to determine the risk of a patient associated with HF, e.g., having an HFH event or other HF event, and adjusting the algorithm on the basis of whether there are false determinations of the risk. Clinicians (e.g., physicians, nurses, and other healthcare personnel) typically manage several to hundreds of patients at any given time. In many cases, the course of treatment may occur while patients are away from a clinical or hospital setting. The challenge of treating many patients without frequent personal contact may make it difficult to provide timely treatments, or changes in treatment, to each patient. The amount of time each clinician is required to spend on each patient may be reduced if the clinician is only notified when an algorithm determines there is a high risk of a future HFH or other HF event.

In some situations, remote patients may have one or more medical devices (e.g., implanted or external devices) monitoring and/or treating one or more conditions. These medical devices may be capable of transmitting information, including diagnostic data, via a network or other communication pathways. An algorithm may use the diagnostic data to determine a patient's varying risk of HFH or another HF event. In some cases, the clinician and/or patient may only be notified once a certain risk threshold is met, and the clinician and/or patient need to take corrective action when the patient's risk status is found to be too high.

As described herein, devices and methods may be used to increase specificity and sensitivity of an algorithm used to determine a risk, e.g., in the form of a score or status designation, of an impending HF event based on integrated diagnostic data of the patient. An HF event can include HFH, or HF with worsening symptoms requiring an ER visit or unscheduled HF clinic visit and managed with transient up-titration of oral diuretics, administration of an IV diuretic, ultrafiltration to manage fluid volume, or treatment with nitrates, as examples. In some examples, the algorithm can stratify HF patients at varying risk of HF events. For example, the algorithm can assign a risk status to the patient (e.g., low, medium, and high) based on the diagnostic data collected by one or more medical devices of the patient.

Diagnostic parameters that may be incorporated in the algorithm may incorporate sensed data from one or more implantable or external medical devices (e.g., patient data). For example, an implantable medical device (IMD) (e.g., a pacemaker, cardioverter and/or defibrillator, or a monitor that does not provide therapy) may automatically generate and store patient data and information used by the algorithm to assign a risk status to the patient. Although diagnostic parameters may include sensed data from a device, the diagnostic parameters may also incorporate patient history or other patient information (as described herein) entered by the clinician or created by another device.

In some examples, when a patient's risk status is found to be high, e.g., due to a risk level or score exceeding a threshold, a clinician is notified via a networked monitoring system to take corrective action. The algorithm, e.g., various thresholds used by the algorithm to classify the patient condition, may be set at certain values based on an analysis of diagnostic data and corresponding patient conditions for a large population of patients. However, a population-based algorithm may have false alerts for a particular patient, such as false negatives or false positives. If too many false positive alerts are triggered by the algorithm for a patient, an unnecessary burden can be placed on the clinicians and the healthcare system. In addition, if the algorithm is not adequately sensitive to detect a high-risk of HFH or another HF event, then the algorithm may also be ineffective because it does provide the necessary alerts to a clinician or patient.

The algorithm may initially be a population level algorithm set for an average HF patient. As diagnostic data and information is collected, the algorithm may be adjusted according to the individual patient based on the detection of false alerts or missed HF events. In addition, the diagnostic data collected from the patient may be periodically used to update the population level algorithm. An electronic medical record (EMR) data system, e.g., data from the system indicating whether a patient experienced an HF event, can be used to verify whether the algorithm correctly predicted that the patient experienced an HF event.

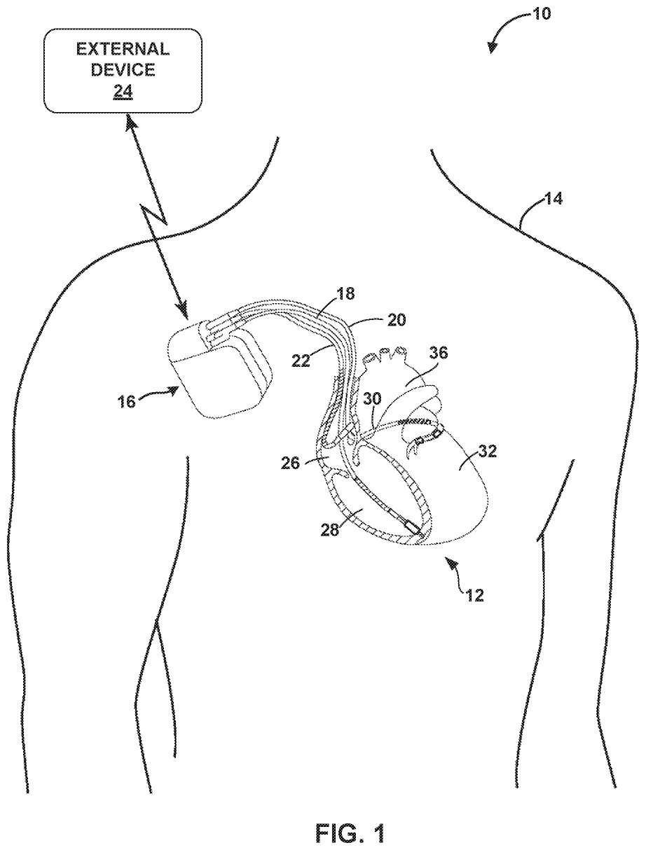

FIG. 1 is a conceptual drawing illustrating an example system 10 configured to collect and transmit patient data from patient 14 for generation of a patient management report. In the example of FIG. 1, system 10 includes IMD 16, which is coupled to leads 18, 20, and 22 and external device 24. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

Although an IMD and delivery of electrical stimulation to heart 12 are described herein as examples, the techniques for generating diagnostic parameters using sensed patient data and other information for a HF status algorithm may be applicable to other medical devices and/or other therapies. In general, the techniques described in this disclosure may be implemented by any medical device, e.g., implantable or external, that senses a signal indicative of cardiac activity, patient activity, or some other physiological or anatomical activity of patient 14. As one alternative example, the techniques described herein may be implemented in an implanted or external cardiac monitor that generates electrograms of heart 12 and detects thoracic fluid volumes, respiration, and/or cardiovascular pressure of patient 14.

In the example of FIG. 1, leads 18, 20, 22 extend into the heart 12 of patient 14 to sense electrical activity of heart 12 and/or deliver electrical stimulation to heart 12. Leads 18, 20, and 22 may also be used to detect a thoracic impedance indicative of fluid volume in patient 14, respiration rates, sleep apnea, electrical events, or other sensed data used to determine the diagnostic parameters. Respiration parameters, e.g., respiration rates, tidal volume, respiration rate and/or volume variability, and sleep apnea, may also be detectable via an electrogram, e.g., based on a signal component in a cardiac electrogram that is associated with respiration. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, system 10 may additionally or alternatively include one or more leads or lead segments (not shown in FIG. 1) that deploy one or more electrodes within the vena cava, other veins, or other locations within one or more heart chambers, the coronary vasculature, or the cardiovascular system. In some examples, the electrodes may be deployed in proximity of the sympathetic nerve plexus located around the coronaries and the concavity of aortic arch. Electrodes located in proximity nerves may facilitate sensing of nerve activity, e.g., sympathetic nerve activity, and/or neurostimulation. Furthermore, in some examples, system 10 may additionally or alternatively include temporary or permanent extravascular, e.g., epicardial or subcutaneous, leads with electrodes implanted outside of heart 12, instead of or in addition to transvenous, intracardiac leads 18, 20 and 22. Such leads may be used for one or more of cardiac or other electrical (e.g., nerve activity) sensing, pacing, or cardioversion/defibrillation. For example, these electrodes may allow alternative electrical sensing configurations that provide improved or supplemental sensing in some patients. In some examples, these other leads may be used to detect intrathoracic impedance for a diagnostic parameter related to HF risk or fluid retention levels.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may detect arrhythmia of heart 12, such as tachycardia or fibrillation of the atria 26 and 36 and/or ventricles 28 and 32, and may also provide defibrillation therapy and/or cardioversion therapy via electrodes located on at least one of the leads 18, 20, 22. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In addition, IMD 16 may monitor the electrical signals of heart 12 for one or more diagnostic parameters used in monitoring patient 14, treating patient 14, and/or generating the patient management report. IMD 16 may utilize two of any electrodes carried on leads 18, 20, 22 to generate electrograms of cardiac activity. In some examples, IMD 16 may also use a housing electrode of IMD 16 (not shown) to generate electrograms and monitor cardiac activity. Although these electrograms may be used to monitor heart 12 for potential arrhythmias and other disorders for therapy, the electrograms may also be used to monitor the condition of heart 12. For example, IMD 16 may monitor heart rate (night time and day time), heart rate variability, ventricular or atrial intrinsic pacing rates, indicators of blood flow, pulse transit time (PTT), or other indicators of the ability of heart 12 to pump blood or the progression of HF.

PTT is the time a pulse resulting from cardiac contraction takes to travel between two points, e.g., a relatively central point and relatively peripheral point, of the cardiovascular system. A shorter transit time can indicate stiffer vessels and elevated blood pressure. In some examples, IMD 16 and/or other devices or processing circuitry of system 10 may monitor PTT to determine vascular tone/relative blood pressure, or changes therein over time. Sensors and techniques for monitoring PTT may include any combination of one or more of electrograms or impedance determined using electrodes, optical signals sensed via optical sensors, or pressure signals sensed via pressure waveforms. Sensors and techniques for monitoring PTT may include those described in commonly-assigned U.S. Patent Application Publication No. 2018/0055386 by Zielinski et al., entitled “SYSTEMS AND METHODS FOR MONITORING HEMODYNAMIC STATUS,” which published on Mar. 1, 2018, and which is incorporated herein by reference in its entirety. PTT, e.g., statistical or trend values of PTT, may be a diagnostic parameter to which an algorithm is applied to determine an HF risk score or status according to the techniques described herein.

In some examples, IMB 16 may also use any two electrodes of leads 18, 20, and 22 or the housing electrode to sense the intrathoracic impedance of patient 14. As the tissues within the thoracic cavity of patient 14 increase in fluid content, the impedance between two electrodes may also change. For example, the impedance between an RV coil electrode and the housing electrode may be used to monitor changing intrathoracic impedance.

IMD 16 may use this impedance to create a fluid index. As the fluid index increases, more fluid is being retained within patient 14 and heart 12 may be stressed to keep up with moving the greater amount of fluid. Therefore, this fluid index may be used for a diagnostic parameter. By monitoring the fluid index in addition to other sensed values, IMD 16 may be able to reduce the number of false positive HF identifications relative to what might occur when monitoring only one or two values indicative of the patient condition. An example system for measuring thoracic impedance and determining a fluid index is described in U.S. Patent Publication No. 2010/0030292 by Sarkar et al., titled, “DETECTING WORSENING HEART FAILURE BASED ON IMPEDANCE MEASUREMENTS,” which published on Feb. 4, 2010 and is incorporated herein by reference in its entirety.

IMB 16 may transmit sensed data or other patient data stored on IMD 16 to external device 24. IMB 16 may transmit the data in response to receiving a request from external device 24, in response to obtaining patient data, when a communication channel is established between IMB 16 and external device 24, or according to a predetermined schedule (e.g., hourly, daily, or weekly). For example, IMB 16 may transmit electrical signals (e.g., an electrocardiogram (ECG)) detected from heart 12, electrical events identified from the ECG (e.g., ventricular or atrial tachycardia, or ventricular or atrial fibrillation), patient activity values, heart rate, respiration rate, impedance values, PTT values, or any other sensed data or information generated from the sensed data. In addition, IMD 16 may transmit information regarding treatment delivered to patient 14. For example, IMD 16 may transmit information regarding shocks delivered to patient 14 (e.g., the number of shocks, parameters of each shock, and timeline of each shock), pacing parameters, stimulation parameters, drug delivery parameters, or any other information related to therapies delivered by IMD 16. The data or information transmitted by IMD 16 may be directed to any data obtained by IMD 16 or only information used to generate the diagnostic parameters used in the algorithm. IMD 16 may automatically sense values from one or more parameters and store them within the IMD for later transmission.

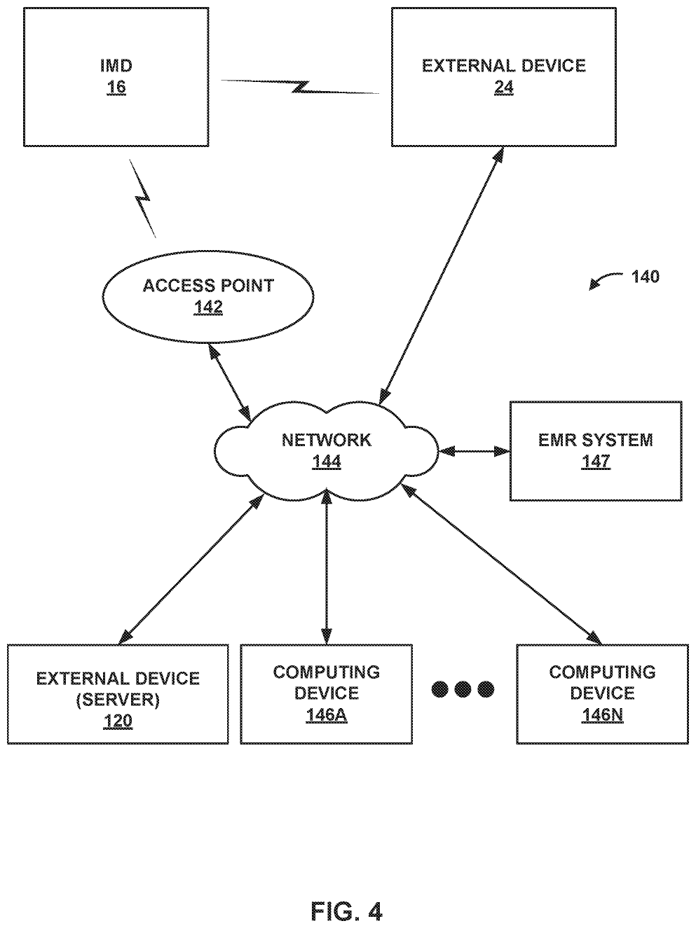

In some examples, the external device 24 may retrieve information from IMD 16 regarding the performance or integrity of IMD 16 or other components of system 10, such as leads 18, 20 and 22, or a power source of IMD 16. This data regarding the function of IMD 16 may be transmitted in raw form and/or processed for use in generating one or more diagnostic parameters. IMD 16 may transmit this information as it is obtained, as it is requested by external device 24, or according to a predetermined transmission schedule. External device 24 may transmit any information received from IMD 16 to a remote sever via a network for processing, analysis, applying the HF monitoring algorithm, and/or presentation to a clinician. In other examples, a home monitor different from external device 24 or other access point (e.g., access point 142 of FIG. 4) may receive information from IMD 16 and/or transmit the information to a server (e.g., server 120 of FIG. 4).

External device 24 may also allow the user to define how IMD 16 senses, detects, and/or manages patient diagnostic parameters. For example, the user may define the frequency of sampling or the evaluation window used to monitor the various values from sensed parameters. In some examples, the user may use external device 24 to select which diagnostic parameters are desired to be sent, characteristics of each diagnostic parameter evaluated, or set respective thresholds against which values for one or more diagnostic parameters are compared. In this manner, external device 24 may be used to customize how diagnostic parameters are generated and when diagnostic parameters are used in the HF monitoring algorithm. In other examples, the user (e.g., a clinician) may use an alternative computing device in communication with a server to manage diagnostic parameters, determine how each diagnostic parameter is calculated, select reporting characteristics for each diagnostic parameter, and/or set thresholds for each diagnostic parameter.

Patient data or information not generated from IMD 16 or another implanted device may also be incorporated into a diagnostic parameter. Patient data from an IMD may include therapy use statistics (e.g., pacing or shocks), heart rate, heart rate variability, patient activity, atrial arrhythmias, impedance data, PTT data, and cardiac resynchronization therapy statistics. Cardiac resynchronization therapy statistics may include, for example, an amount e.g., percentage, of cardiac beats during which CRT is delivered and/or for which CRT is delivered and results in effective therapy to synchronize the ventricular contraction (e.g., the EffectivCRT™ diagnostic commercially available from Medtronic plc of Dublin, Ireland.

Patient information may include clinic history (e.g., procedures, therapies, diagnoses, and/or laboratory results), therapy history (e.g., detailed instances of delivered therapy, dosages of therapies, parameters defining therapy, and/or results of delivered therapies), and patient condition status (e.g., current patient conditions indicated by device collected values or clinician observed values). This information (e.g., non-device information) may also include patient history, patient entered information (e.g., responses to questionnaires or entries into a patient diary), clinician entered information, or other information related to the patient or patient condition such as those collected using standardized instruments (e.g. Minnesota Living with HF Questionnaire from the University of Minnesota, for use in controlled clinical trials designed to test superiority or non-inferiority of medical devices in support of regulatory submissions; Kansas City Cardiomyopathy Questionnaire (KCCQ-12), a self-administered instrument that measures patient-reported symptoms, function and quality of life for patients with heart failure; and PHQ-9, a 9-question instrument given to patients in a primary care setting to screen for the presence and severity of depression).

Example information may further include patient weight, medication compliance, consumed food, liquid intake, activity durations, pain levels, pain locations, urinary or fecal voiding events, durations of treatment, allergies, psychological status, or any other patient information related to the diagnosis and treatment of a patient including non-device information that may describe or otherwise characterize the health of patient 14. The non-device information may be stored in IMD 16, external device 24, a remote repository via a network, or other database.

IMD 16 and external device 24 may communicate via wireless communication using any techniques known in the art. An example communication technique is radiofrequency (RF) telemetry, e.g., via Bluetooth® or a proprietary RF communication protocol, but other communication techniques such as magnetic coupling are also contemplated. In some examples, external device 24 may include a programming head that may be placed proximate to the body of the patient near the IMD 16 implant site in order to improve the quality or security of communication between IMD 16 and external device 24.

The patient data collected from IMD 16 and/or other patient information may be synthesized to generate one or more diagnostic parameters, e.g., values of diagnostic parameters may be computed based on the patient data collected from IMD 16 and/or other patient information. Each diagnostic parameter may be used to indicate one or more changes in the patient condition or events that the patient has endured. In this manner, the diagnostic parameters may be used by the clinician to change a course of treatment for a patient. In addition, each diagnostic parameter may alone, or in combination be indicative of HF of patient 14.

IMD 16 may automatically detect a plurality of diagnostic parameters of patient 14, also referred to as patient parameters. These parameters may alone, or in combination, be indicative of HF status of patient 14. The patient parameters may include, as examples, thoracic impedance, heart rate variability, the number, frequency or duration of atrial fibrillation after cardioversion therapy, ventricular rate during persistent atrial fibrillation, day and night heart rate, or any other parameters detectable from patient 14 or based on the treatment of patient 14.

As one example, a HF risk score may indicate a high risk of an HF event when a predetermined number of the plurality of automatically detected patient parameters, such as two or more automatically detected patient parameters, each meet their respective parameter-specific threshold. As another example, the HF risk score may indicate a medium risk of an HF event when a predetermined number of the plurality of automatically detected patient parameters, such as only one automatically detected patient parameter, meets its respective parameter-specific threshold. In an additional example, the HF risk score may indicate a low risk of an HF event when none of the plurality of automatically detected patient parameters meets their respective parameter-specific thresholds. An algorithm can be used to compare the parameter values to the parameter thresholds and determine the HF risk score, as described herein.

IMD 16 may automatically detect and/or determine values of each of the patient parameters and store them within the IMD for later transmission. Although IMD 16 may automatically detect eight different patient parameters in some examples, IMD 16 may detect more or less patient parameters in other examples. For example, the patient parameters may include two or more of a thoracic fluid index, an atrial fibrillation duration, a ventricular contraction rate during atrial fibrillation, a patient activity, a nighttime heart rate, a heart rate variability, a cardiac resynchronization therapy (CRT) statistic (e.g., the percentage of cardiac cycles for which 14 cardiac resynchronization pacing was provided), or the occurrence of or number of therapeutic electrical shocks.

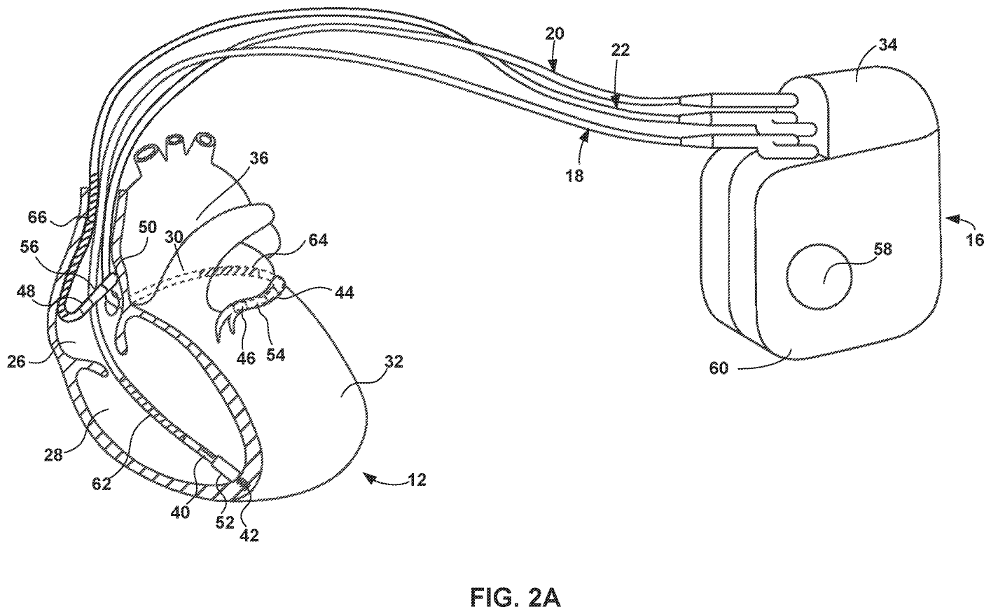

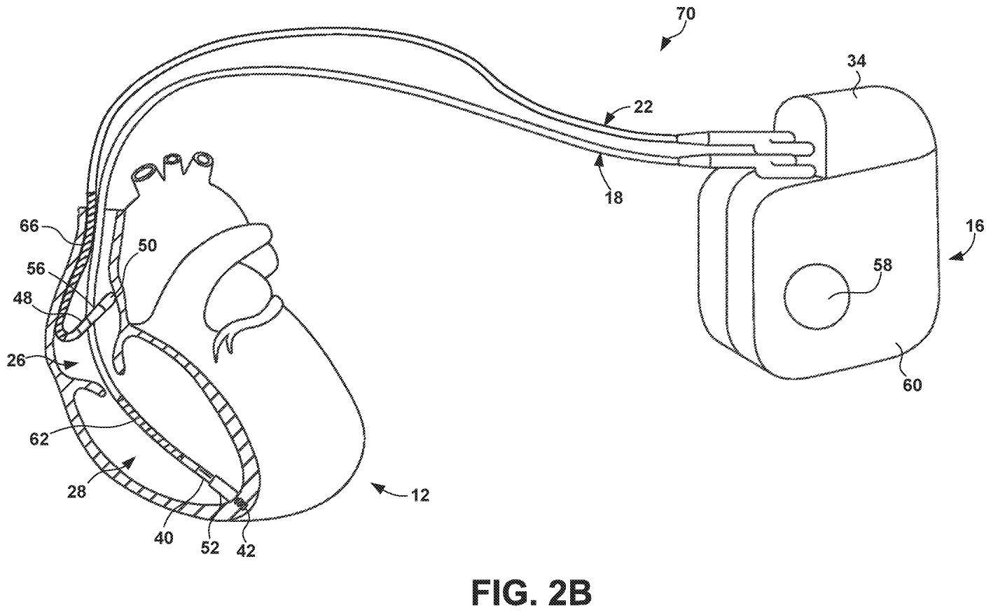

FIG. 2A is a conceptual drawing illustrating IMD 16 and leads 18, 20, and 22 of system 10 in greater detail. As shown in FIG. 2A, IMD 16 is coupled to leads 18, 20, and 22. Leads 18, 20, 22 may be electrically coupled to signal generation circuitry, e.g., stimulation generator, and a sensing circuitry of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44, and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

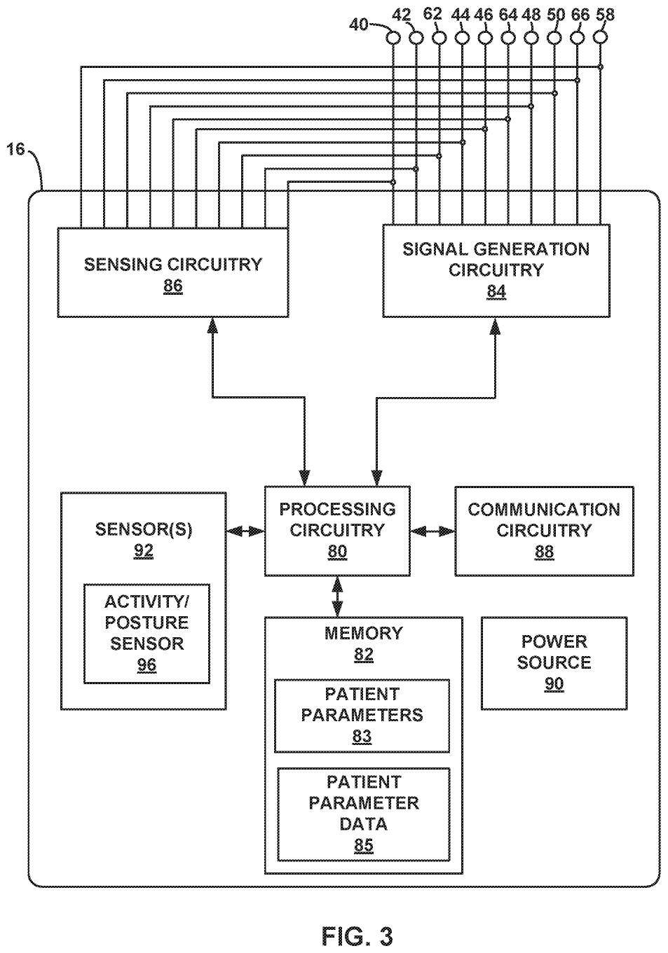

In some examples, as illustrated in FIG. 2A, IMD 16 includes one or more housing electrodes, such as housing electrode 58, which may be formed integrally with an outer surface of hermetically-sealed housing 60 of IMD 16, or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 3, housing 60 may enclose signal generation circuitry that generates therapeutic signals, such as cardiac pacing pulses and defibrillation shocks, as well as a sensing circuitry for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration or electrode vector.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of stimulation or sensing, their associated conductors and connectors, and any tissue or fluid between the electrodes, may define an electrical path.

The configuration of system 10 illustrated in FIGS. 1 and 2A is merely one example. In other examples, a system may include epicardial leads and/or subcutaneous electrodes instead of or in addition to the transvenous leads 18, 20, 22 illustrated in FIG. 1. Further, IMD 16 need not be implanted within patient 14. In examples in which IMD 16 is not implanted in patient 14, IMD 16 may sense electrical signals and/or deliver defibrillation pulses and other therapies to heart 12 via percutaneous leads that extend through the skin of patient 14 to a variety of positions within or outside of heart 12. Further, external electrodes or other sensors may be used by IMD 16 to deliver therapy to patient 14 and/or sense and detect values of patient parameters used to generate one or more diagnostic parameters for patient 14.

In addition, in other examples, a system may include any suitable number of leads coupled to IMD 16, and each of the leads may extend to any location within or proximate to heart 12. For example, systems in accordance with this disclosure may include three transvenous leads located as illustrated in FIGS. 1 and 2A, and an additional lead located within or proximate to left atrium 36. As another example, systems may include a single lead that extends from IMD 16 into right atrium 26 or right ventricle 28, or two leads that extend into a respective one of the right atrium 26 and right ventricle 28. An example of a two-lead system is shown in FIG. 2B. Any electrodes located on these additional leads may be used in sensing and/or stimulation configurations.

In some examples, a system may additionally or alternatively include one more subcutaneous pacing and/or defibrillation devices coupled to extravascular leads, leadless pacing devices configured for implantation within a heart chamber, and/or implantable or external monitoring devices that monitor patient parameters but do not provide therapy, such as the Reveal LINQ™ insertable cardiac monitor. Any such devices may be configured to determine diagnostic parameter values for consideration by an HF algorithm as described herein.

Any of electrodes 40, 42, 44, 46, 48, 50, 62, 64, 66, and 58 may be utilized by IMD 16 to sense or detect signals used to determine diagnostic parameter values for patient 14. IMD 16 may detect and collect patient data from those electrode vectors used to treat patient 14. For example, IMD 16 may derive an atrial fibrillation duration, heart rate, heart rate variability, and PTT value from electrograms generated via electrode vectors used to deliver pacing therapy. However, IMD 16 may utilize other electrodes to detect values for these types of parameters from patient 14 in some examples.

In addition to electrograms of cardiac signals, any of electrodes 40, 42, 44, 46, 48, 50, 62, 64, 66, and 58 may be used to sense non-cardiac signals. For example, two or more electrodes may be used to measure an impedance within the thoracic cavity of patient 14. This intrathoracic impedance may be used to generate a fluid index parameter that indicates the amount of fluid building up within patient 14. Since a greater amount of fluid may indicate increased pumping loads on heart 12, the fluid index may be used as an indicator of HF risk. IMD 16 may periodically measure the intrathoracic impedance to identify a trend in the fluid index over days, weeks, months, and even years of patient monitoring.

In general, the two electrodes used to measure the intrathoracic impedance may be located at two different positions within the chest of patient 14. For example, coil electrode 62 and housing electrode 58 may be used as the sensing vector for intrathoracic impedance because electrode 62 is located within RV 28 and housing electrode 58 is located at the IMD 16 implant site generally in the upper chest region. However, other electrodes spanning multiple organs or tissues of patient 14 may also be used, e.g., an additional implanted electrode used only for measuring thoracic impedance.

As another example of a non-cardiac signal, any of electrodes 40, 42, 44, 46, 48, 50, 62, 64, 66, and 58 may be used to sense nerve signals. In some examples, as discussed above, these or other electrodes may be positioned proximate to a sympathetic nerve plexus located around the coronary vasculature and the concavity of the aortic arch. Sympathetic nerve activity may change with changes to patient cardiovascular health, including the HF status of the patient. As such, statistical values and/or trends derived from sensed sympathetic nerve activity may be a diagnostic parameter to which an algorithm is applied to determine an HF risk score or status according to the techniques described herein. Sensing circuitry and/or processing circuitry of system 10, e.g., of IMD 16, may be configured differently to detect cardiac electrogram signals, neural signals, and impedance. For examples, different amplification, filtering, and feature detection techniques may be used in each case.

FIG. 2B is a conceptual diagram illustrating another example system 70, which is similar to system 10 of FIGS. 1 and 2, but includes two leads 18, 22, rather than three leads. Leads 18 and 22 are implanted within right ventricle 28 and right atrium 26, respectively. System 70 shown in FIG. 2B may be useful for physiological sensing and/or providing pacing, cardioversion, or other therapies to heart 12. Detection of one or more patient parameters and transmission of patient data according to this disclosure may be performed in two lead systems in the manner described herein with respect to three lead systems. In other examples, a system similar to systems 10 and 70 may only include one lead (e.g., any of leads 18, 20 or 22) to deliver therapy and/or sensor and detect patient parameters related to monitoring risk of HF, arrhythmias, or any other patient condition. Alternatively, diagnostic parameter data may be implemented in systems utilizing extravascular leads, subcutaneous IMDs, or external medical devices. Although FIGS. 1 and 2 provide an exemplary IMD 16 implantation in which the notification differentiation described below may be implemented, it is understood that IMD 16 and its associated electrodes may be implemented in other locations of the patient's body and may include leads or may be leadless.

FIG. 3 is a functional block diagram illustrating an example configuration of IMD 16. In the illustrated example, IMD 16 includes a processing circuitry 80, memory 82, signal generation circuitry 84, sensing circuitry 86, communication circuitry 88, power source 90, and one or more sensor(s) 92. Memory 82 includes computer-readable instructions that, when executed by processing circuitry 80, cause IMD 16 and processing circuitry 80 to perform various functions attributed to IMD 16 and processing circuitry 80 herein. Memory 82 may include any volatile, non-volatile, magnetic, optical, or electrical storage media, such as a random-access memory (RAM), read-only memory (ROM), non-volatile RAM (NVRAM), electrically-erasable programmable ROM (EEPROM), flash memory, or any other digital or analog storage media.

Processing circuitry 80 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 80 may include multiple components, such as any combination of one or more microprocessors, one or more controllers, one or more DSPs, one or more ASICs, or one or more FPGAs, as well as other discrete or integrated logic circuitry. The functions attributed to processing circuitry 80 herein may be embodied as software, firmware, hardware or any combination thereof. Processing circuitry 80 may control signal generation circuitry 84 to deliver therapy to heart 12 according to therapy parameters, which may be stored in memory 82. For example, processing circuitry 80 may control signal generation circuitry 84 to deliver electrical pulses with the amplitudes, pulse widths, frequency, or electrode polarities specified by the therapy parameters.

Signal generation circuitry 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, signal generation circuitry 84 is configured to generate and deliver therapeutic electrical signals to heart 12. For example, signal generation circuitry 84 may deliver defibrillation shocks to heart 12 via at least two electrodes 58, 62, 64, 66. Signal generation circuitry 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generation circuitry 84 delivers pacing, cardioversion, or defibrillation therapy in the form of electrical pulses. In other examples, signal generation circuitry 84 may deliver one or more of these types of therapy in the form of other signals, such as sine waves, square waves, or other substantially continuous time signals.

Signal generation circuitry 84 may include voltage and/or current regulation circuitry for generating voltages and currents, charge pump circuitry and energy storage circuitry for generating and storing charge, and switching circuitry. Processing circuitry 80 may use the switching circuitry to control the timing of therapeutic signals to select, e.g., via a data/address bus, which of the available electrodes are used to deliver the signals and the polarities of the electrodes. The switching circuitry may include a switch array, switch matrix, multiplexer, or any other type of switching device suitable to selectively couple signals to selected electrodes.

Sensing circuitry 86 monitors signals from at least one of electrodes 40, 42, 44, 46, 48, 50, 58, 62, 64 or 66 in order to monitor electrical activity of heart 12, impedance, nerve activity, or other electrical phenomenon. Cardiac electrogram sensing may be done to determine heart rates or heart rate variability, to detect arrhythmias or other cardiac electrical signals, and to determine the starting time of a PTT measurement. Based on impedances sensed via sensing circuitry 86, processing circuitry may determine a fluid index, as described herein, one or more respiration parameters, such as rate, magnitude (e.g., volume), or variability of rate or magnitude, or determine the ending time of a PTT measurement.

Sensing circuitry 86 may also include switching circuitry to select which electrode vector or combination, e.g., which of the available electrodes and their polarities, are used to sense the heart activity or impedance. In some examples, processing circuitry 80 may select the electrodes that function as sense electrodes, i.e., select the sensing configuration, via the switching circuitry. The switching circuitry of sensing circuitry 86 may include a switch array, switch matrix, multiplexer, or any other type of switching device suitable to selectively couple signals to selected electrodes, and may be the same as and/or different than the switching circuitry of signal generation circuitry 84. Sensing circuitry 86 may include one or more detection channels, each of which may be coupled to a selected electrode configuration for detection of cardiac signals via that electrode configuration. Some detection channels may be configured to detect cardiac events, such as P- or R-waves, and provide indications of the occurrences of such events to processing circuitry 80.

Processing circuitry 80 may include a timing and control module, which may be embodied as hardware, firmware, software, or any combination thereof. The timing and control module may comprise a dedicated hardware circuit, such as an ASIC, separate from other processing circuitry 80 components, such as a microprocessor, or a software module executed by a component of processing circuitry 80, which may be a microprocessor or ASIC. The timing and control module may implement programmable counters. If IMD 16 is configured to generate and deliver pacing pulses to heart 12, such counters may control the basic time intervals associated with DDD, VVI, DVI, VDD, AAI, DDI, DDDR, VVIR, DVIR, VDDR, AAIR, DDIR, CRT, and other modes of pacing.

Intervals defined by the timing and control module within processing circuitry 80 may include atrial and ventricular pacing escape intervals, refractory periods during which sensed P-waves and R-waves are ineffective to restart timing of the escape intervals, and the pulse widths of the pacing pulses. As another example, the timing and control module may withhold sensing from one or more channels of sensing circuitry 86 for a time interval during and after delivery of electrical stimulation to heart 12. The durations of these intervals may be determined by processing circuitry 80 in response to stored data in memory 82. The timing and control module of processing circuitry 80 may also determine the amplitude of the cardiac pacing pulses.

Interval counters implemented by the timing and control module of processing circuitry 80 may be reset upon sensing of R-waves and P-waves with detection channels of sensing circuitry 86. In examples in which IMD 16 provides pacing, signal generation circuitry 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. In such examples, processing circuitry 80 may reset the interval counters upon the generation of pacing pulses by signal generation circuitry 84, and thereby control the basic timing of cardiac pacing functions, including anti-tachyarrhythmia pacing.

The value of the count present in the interval counters when reset by sensed R-waves and P-waves may be used by processing circuitry 80 to measure the durations of R-R intervals, P-P intervals, P-R intervals and R-P intervals, which are measurements that may be stored in memory 82. Processing circuitry 80 may use the count in the interval counters to detect a tachyarrhythmia event, such as atrial fibrillation (AF), atrial tachycardia (AT), ventricular fibrillation (VF), or ventricular tachycardia (VT). These intervals may also be used to detect the overall heart rate, ventricular contraction rate, and heart rate variability. A portion of memory 82 may be configured as a plurality of recirculating buffers, capable of holding series of measured intervals, which may be analyzed by processing circuitry 80 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. The tachyarrhythmia detection algorithms may be based on the ventricular and/or atrial depolarization rate, the regularity of the depolarization rate, e.g., on a beat-to-beat or other basis, and or the morphology of one or more cardiac electrograms. However, other arrhythmia detection methodologies may also be employed by processing circuitry 80 in other examples.

In some examples, processing circuitry 80 may determine that tachyarrhythmia has occurred by identification of shortened R-R (or P-P) interval lengths. Generally, processing circuitry 80 detects tachycardia when the interval length falls below 220 milliseconds (ms) and fibrillation when the interval length falls below 180 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In the event that processing circuitry 80 detects an atrial or ventricular tachyarrhythmia based on signals from sensing circuitry 86, and an anti-tachyarrhythmia pacing (ATP) regimen is desired, timing intervals for controlling the generation of ATP therapies by signal generation circuitry 84 may be loaded by processing circuitry 80 to control the operation of the escape interval counters and to define refractory periods during which detection of R-waves and P-waves is ineffective to restart the escape interval counters for the ATP. In the event that processing circuitry 80 detects an atrial or ventricular tachyarrhythmia based on signals from sensing circuitry 86, and a cardioversion or defibrillation shock is desired, processing circuitry 80 may control the amplitude, form and timing of the shock delivered by signal generation circuitry 84.

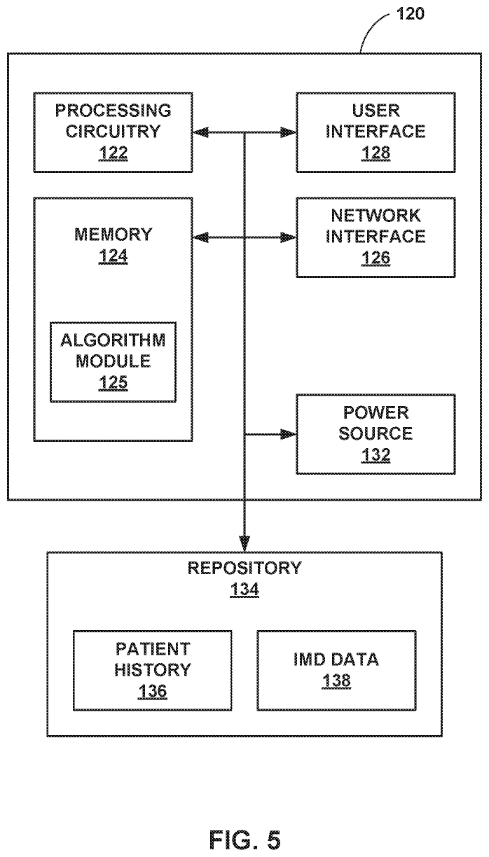

Memory 82 may be configured to store a variety of operational parameters, therapy parameters, sensed and detected data, instructions for processing circuitry 80 related to determining diagnostic parameters based on the sensed and detected data, and any other information related to the therapy and treatment of patient 14. In the example of FIG. 4, memory 82 also includes patient parameters 83 and patient parameter data 85. Patient parameters 83 may include all of the parameters and instructions required by processing circuitry 80 and sensor 92 to sense and detect each of the patient parameters used to generate the diagnostic information transmitted by IMD 16, e.g., to external device 24 (FIG. 1) or external device 120 (FIGS. 4 and 5) via communication circuitry 88. Patient parameter data 85 may store all of the data generated from the sensing and detecting of each patient parameter. Patient parameter data 85 may store all of the data generated from the sensing and detecting of each patient parameter. In this manner, memory 82 stores a plurality of automatically detected patient parameters as the data required to generate a risk status of patient 14 being admitted to the hospital due to HF.

Sensors 92 may be configured to, e.g., include transducers and other circuitry configured to, sense any of the patient parameters described herein. Sensors 92 may include processing circuitry that is different than, or at least partially overlapping with, processing circuitry 80, and may be embodied as software or firmware executed by such processing circuitry. In some examples, sensors 92 may include a software/firmware module executed by processing circuitry 80. In some examples, sensors 92 may include one or more activity/posture sensors 96 configured to sense an activity level, posture, and/or falls of patient 14, e.g., frequency, severity, and or risk of falls, such as one or more accelerometers, e.g., a multi-axis accelerometer. Diagnostic parameters to which an algorithm is applied to determine an HF risk score or status according to the techniques described herein may include: activity metrics, such as daily average level, daytime average level, amount of time above a threshold activity level, and response heart rate to activity level; posture metrics, such as nighttime or sleeping posture, daytime or awake posture, or amount of postural changes during sleep or awake periods; and fall metrics, such as number of falls over a given duration (e.g., six months or one year) and/or severity.

In some examples, memory 82 may include separate memories to store instructions for processing circuitry 80 to generate desired patient data, generated patient data, patient history generated by IMD 16 or received from an external device, false positive or false negative HF events associated with algorithm, or any other distinct types of data. Memory 82 may retain patient data once transmitted to an external device or delete patient data once the data has been transmitted.

Patient parameters 83 may include definitions of each of the patient parameters sensed or measured by sensor 92 and sensing circuitry 86. These definitions may include instructions regarding what electrodes or sensors to use in the detection of each parameter, the sample rate, calibration schemes, parameter-specific thresholds, and any other related information. In one example, patient parameters 83 may include such information for a thoracic fluid index (or another parameter determined based on thoracic impedance), an atrial tachycardia or fibrillation burden, a ventricular contraction rate during atrial fibrillation, a patient activity, a nighttime heart rate, a difference between night and day heart rate, a heart rate variability, a cardiac resynchronization therapy percentage, a bradyarrhythmia pacing therapy percentage (in a ventricle and/or atrium), and number or frequency of electrical shock events. In other examples, other patient parameters may be stored that may be useful in the detection of HF risk, e.g., blood pressure, right ventricular pressure, pulmonary artery pressure, patient temperature, lung volume, lung tidal volume, lung density, breathing rate or even biomarkers such as a brain natriuretic peptide (BNP), troponin, or related surrogates. In such examples, IMD 16 may include or be coupled to sensors known in the art for detecting such parameters. In some examples, the atrial tachycardia or fibrillation burden may be a time of the event, a percent or amount of time over a certain period, a number of episodes, or even a frequency of episodes.

In examples in which a portion of an HF algorithm that includes comparison of patient parameter values to a parameter-specific threshold is performed by IMD 16, patient parameters 83 may also store the parameter-specific threshold for each of the patient parameters. In some examples, an external device, e.g., external device 24 (FIG. 1) or 120 (FIGS. 4 and 5) compares the patient parameter values to parameter-specific thresholds. Parameter thresholds may be predetermined and held constant over the entire monitoring of patient 14. In some examples, however, parameter thresholds may be modified by a user during therapy or processing circuitry 80 to compensate for certain patient conditions. For example, a heart rate threshold may be changed over the course of monitoring if the normal or baseline heart rate has changed during therapy.

In one example, these parameter-specific thresholds may include a thoracic fluid index threshold of approximately 60, an atrial fibrillation burden threshold of approximately 6 consecutive hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 40 milliseconds for seven consecutive days, a cardiac resynchronization therapy percentage threshold of 90 percent for five of seven consecutive days, and an electrical shock number threshold of 1 electrical shock. These thresholds may be different in other examples, and may be configured by a user, e.g., a clinician, for an individual patient.

Processing circuitry 80 and/or processing circuitry of another computing device, e.g., external device 24 (FIG. 1) or external device 120 (FIG. 4) may determine a HF risk score and/or status that is based on the patient parameters and whether any of the parameters meet their respective thresholds. Any time that an automatically detected patient parameter meets their respective parameter threshold, the patient threshold may be counted in the risk score. In one example, if two or more of the eight patient parameters meet their respective parameter threshold, then the risk status would be classified as a high risk. In other examples, the risk score may include a numerical value such as 2 out of 8 (e.g., threshold meeting parameters out of the total number of monitored parameters). In another embodiment, the parameters may be combined using a probabilistic approach such as Bayesian Belief Network. One example of such a statistical analysis is described in PCT Patent Publication No. WO 2011/126823A1 titled, “METHOD AND APPARATUS FOR MONITORING TISSUE FLUID CONTENT FOR USE IN AN IMPLANTABLE CARDIAC DEVICE,” which is incorporated by reference herein in its entirety. The higher the risk score, the more likely that patient 14 is at risk to have an HF event and/or be admitted to the hospital within a predefined number of days. For example, each threshold meeting parameter counted in the predetermined number may contribute to a higher risk score of HFH or another HF event. In this example, a risk score of 1 out of 8 may indicate a medium risk of an HF event, a risk score of 2 out of 8 may indicate a high risk of an HF event, and a risk score of 3 out of 8 may indicate a very high risk of an HF event.

Meeting a parameter threshold does not require that the detected value of the patient parameter becomes greater than the magnitude of the threshold. For some patient parameters, meeting the parameter threshold may occur when the value of the patient parameter drops below the parameter threshold. Therefore, each threshold may be a boundary that triggers the parameters inclusion in the HF risk score any time that the parameter threshold is crossed. In other examples, as described above, the risk score may be calculated as a sum of weighted parameters such that some parameters may impact the risk score greater than other parameters (e.g., a trans-thoracic impedance may be weighted double that of other parameters.

In some examples, the algorithm may apply one parameter-specific threshold per patient parameter, but other examples may include several thresholds to apply depending on other patient conditions, delivered therapies, or even the importance of one patient parameter. For example, the thoracic fluid index determined from sensed intrathoracic impedance may be subject to two separate parameter thresholds each counting towards the predetermined number of the HF risk score. The first thoracic fluid index threshold may be set to a value of 60 ohm-days, but the second thoracic fluid index threshold may be set to a value of 100 ohm-days. If the thoracic fluid index parameter meets the first thoracic fluid index threshold of 60 ohm-days, the fluid index parameter may be counted in the HF risk score. If the fluid index also crosses the second thoracic fluid index threshold of 100 ohm-days, the fluid index parameter may be counted in the HF risk score a second time. In this manner, the HF risk score may weight more extreme values of some parameters more heavily than other parameters. In one example, the fluid index value may be a unitless number using a recent intrathoracic impedance, a short term mean impedance, an impedance variability value, and a duration value. Example fluid index values and impedance measurements are described in the above-referenced U.S. Patent Publication No. 2010/0030292 to Sarkar et al.

As the intrathoracic impedance remains low, the fluid index may increase. Conversely, as the intrathoracic impedance remains high, the fluid index may decrease. In this manner, the fluid index value may be a numerical representation of retained fluid that is specific to patient 14. In other examples, the intrathoracic impedance may be alternatively used.

In some examples, as discussed above, a statistical or probability analysis may be performed by the algorithm on some or all of the patient parameters to determine the probability that patient 14 will be hospitalized for HF or experience another HF event, e.g., as described in the above-referenced PCT Patent Publication No. WO 2011/126823A1. In some examples, the HF risk score may be determined without utilizing thresholds for each of the detected patient parameters. Instead, processing circuitry may examine the values of each of the patient parameters for relative contributions to the possibility that patient 14 is at a higher risk of being hospitalized. For example, a Bayesian Belief Network may use the values of the patient parameters to determine the risk score that patient 14 will experience an HF event.

In some examples, a high risk status can have approximately 10-fold higher risk of HF hospitalization compared to the low risk in the next 30 days and approximately 3-fold higher risk compared to the medium risk in the next 30 days. The positive predictive value (PPV) of high risk status with HF hospitalization as an endpoint (e.g., proportion of high status that would be followed by a HF hospitalization within the next 30 days) is in the range between about 6% to about 10%. The PPV increases to approximately 65% if the endpoint considered is worsening signs and symptoms of HF. Furthermore, PPV increases to approximately 85% if the endpoint includes non-compliance with medication, exercise and nutrition in addition to worsening signs and symptoms of HF. This latter endpoint is important as all of its three components (worsening signs, worsening symptoms, and non-compliance) are actionable, thus allowing clinicians to take a corrective action when a HF patient's risk status is found to be high. Despite high PPV of such an algorithm for actionable clinical events, the algorithm can have false positives (in some cases, about 15-35% of the transmissions depending on the endpoint).

Patient parameter data 85 is a portion of memory 82 that may store some or all of the patient parameter data that is determined by processing circuitry 80 based on signals sensed and detected by sensor(s) 92 and sensing circuitry 86. Patient parameter data 85 may store the data for each parameter on a rolling basis during an evaluation window. The evaluation window may only retain recent data and delete older data from the evaluation window when new data enters the evaluation window. In this manner, the evaluation window may include only recent data for a predetermined period of time. Processing circuitry 80 may access parameter data when necessary to retrieve and transmit patient parameter data and/or generate HF risk scores and statuses. In addition, patient parameter data 85 may store HF risk scores or other generated information related to the HF risk of patient 14. Although patient parameters 83 and/or patient parameter data 85 may consist of separate physical memories, these components may be an allocated portion of the greater memory 82 in some examples.

Processing circuitry 80 may determine values of any of the patient parameters used as a basis for generating the HF risk score or otherwise indicating the HF score or status, e.g., the risk at which the patient 14 is for HFH. In one example, processing circuitry 80 (or processing circuitry of an external device) may compare each of the patient parameters to their respective parameter-specific thresholds defined in patient parameters 83 to generate the HF risk score. The processing circuitry may evaluate two or more patient parameters, such as eight different patient parameters.

In one example, processing circuitry 80 may analyze electrograms received from sensing circuitry 86 to detect an atrial fibrillation or atrial tachycardia, and determine atrial tachycardia or fibrillation burden, e.g., duration, as well as a ventricular contraction rate during atrial fibrillation. Processing circuitry 80 may also analyze electrograms in conjunction with a real-time clock, patient posture or activity signal, e.g., from activity/posture sensor 96, and/or other physiological signals indicative of when a patient is asleep or awake to determine a nighttime (or sleeping) heart rate or a daytime (or awake) heart rate or a difference between the day and night heart rate, and also analyze electrograms to determine a heart rate variability, or any other detectable cardiac events from one or more electrograms. As described above, processing circuitry 80 and sensing circuitry 86 may use peak detection, interval detection, or other methods to analyze the electrograms.

In addition, processing circuitry 80 may determine the thoracic impedance used to generate the thoracic fluid index based on impedance detected via a combination of electrodes. As described herein, any of the electrodes of FIGS. 1-3 may be used to take intrathoracic impedance measurements. In other examples, processing circuitry 80 may utilize separate electrodes coupled to IMD 16 or in wireless communication with communication circuitry 88. Once processing circuitry 80 determines the intrathoracic impedance of patient 14, the processing circuitry may generate the thoracic fluid index and compare the index to the thoracic fluid index threshold defined in patient parameters 83.

Activity/posture sensor 96 may include one or more accelerometers or other devices capable of detecting motion and/or position of patient 14. Activity/posture sensor 96 may therefore detect activities of patient 14, postures engaged by patient 14, or falls of patient 14. Processing circuitry 80 may, for example, monitor the patient activity parameter based on the magnitude or duration of each activity and compare the determined parameter data to the activity threshold defined in patient parameters 83. The activity patient parameter may then be used to generate the HF risk score. In addition, the activity patient parameter may also be used in combination with heart rate data to determine a diagnostic parameter indicative of the patient's heart rate response during daily living activity as well as exercise. A blunted heart rate response, also known as chronotropic incompetence, can be a marker of poor health and can indicate a higher risk of an HF event.

In addition to detecting events of patient 14, processing circuitry 80 may also track certain therapies delivered by signal generation circuitry 84, e.g., as directed by processing circuitry 80. Example patient parameters detected by this method may include a CRT parameter (e.g., amount delivered and/or amount effective) or parameters related to delivery of electrical shocks.

The CRT parameter may be the amount or percentage of time each day, or an amount or percentage of cardiac cycles, as examples, that IMD 16 delivers CRT to heart 12 or that IMD 16 delivers effective CRT to heart. Low CRT amounts or percentages may indicate that beneficial therapy is not being delivered and that adjustment of therapy parameters, e.g., an atrioventricular delay or a lower pacing rate, may improve therapy efficacy. In one example, higher CRT amounts or percentages may indicate that heart 12 is sufficiently pumping blood through the vasculature with the aid of therapy to prevent fluid buildup. In examples of other types of cardiac pacing (non-CRT) or stimulation therapy, higher therapy percentages may indicate that heart 12 is unable to keep up with blood flow requirements.

An electrical shock may be a defibrillation event or other high energy shock used to return heart 12 to a normal rhythm. The parameter related to electrical shocks may be a number or frequency of electrical shocks, e.g., a number of shocks within a period of time. Processing circuitry implementing an HF monitoring algorithm may compare these parameters to a CRT percentage and shock event threshold, respectively, to determine when each patient parameter has become critical. In one example, the electrical shock event parameter may become critical when a threshold number of shocks is delivered, e.g., within a time period, or even when patient 14 even receives one therapeutic shock.

In some examples, raw data used to produce patient parameter data may be stored in patient parameter data 85 for later processing or transmission to an external device, e.g., external device 12 (FIG. 1) or external device 120 (FIGS. 4 and 5). An external device may then produce each patient parameter from the raw data, e.g., electrogram or intrathoracic impedance. In some examples, processing circuitry 80 may additionally receive data from one or more implanted or external devices used to detect one or more diagnostic parameters which IMD 16 may store as parameter data in memory 82.

Processing circuitry 80, or processing circuitry of an external device 24 (FIG. 1) or external device 120 (FIGS. 4 and 5) may generate the HF risk score based upon the patient parameters sensed, detected, and stored in patient parameter data 85 of memory 82 of IMD 16. For example, the processing circuitry may continually update the HF risk score as each patient parameter is updated. In other examples, the processing circuitry may periodically update the HF risk score according to an updating schedule. In examples in which processing circuitry of an external device implements the algorithm, the processing circuitry may update the score in response to receiving a new transmission with patient parameter data 85 from IMD 16. In any case, the processing circuitry may compare each of the automatically detected patient parameters to their respective parameter-specific thresholds and automatically generate the HF risk score based on the comparison.

As part of the HF monitoring algorithm, the processing circuitry may also compare the HF risk score to the predetermined threshold risk score stored in memory 82. The predetermined threshold may indicate when patient 14 is at an increased risk of HF. The predetermined threshold may be a percentage or a number of patient parameters meeting their respective parameter threshold. Although a clinician may be presented with the HF risk status at any time, the processing circuitry may push the HF risk status, e.g., when high or critical, to a clinician or other healthcare professional in an alert. This immediacy may be necessary because a critical risk status indicates that HF may be imminent in a large number of patients with the same patient parameter levels. Therefore, a clinician may receive the transmitted diagnostic information of the critical risk status and initiate alternative treatment to prevent patient 14 from hospitalization.

In some examples, external device 24, external device 120, or another computing device or server may implement the algorithm for monitoring for HF events, which may generate the risk score based on diagnostic information, including patient parameter values, transmitted by IMD 16. However, processing circuitry 80 of IMD 16 may still collect and store the data for each patient parameter or even organize and format the patient parameter data before transmitting the patient parameters in patient parameter data 85 to the external device. In addition, processing circuitry 80 may transmit the parameter thresholds with the patient parameter data so that any external device may generate HF risk scores and statuses specific to patient 14, or the external device may store the thresholds.

Communication circuitry 88 includes any suitable hardware, firmware, software or any combination thereof for communicating with another device, such as external device 24 (FIG. 1). In some examples, communication circuitry 88 includes one or more antennae, signal generation and/or oscillation circuitry, and modulation and/or demodulation circuitry to transmitting and receiving signals according to a wireless communication protocol. Under the control of processing circuitry 80, communication circuitry 88 may receive downlink telemetry from and send uplink telemetry to external device 24 with the aid of an antenna, which may be internal and/or external. Processing circuitry 80 may provide the patient parameter data 85 to be uplinked to external device 24 and/or external device 120 and the control signals for the telemetry circuit within communication circuitry 88. In some examples, communication circuitry 88 may provide received data to processing circuitry 80 via a multiplexer.

In some examples, IMD 16 may signal external device 24 to further communicate with and pass the diagnostic parameter data or an HF score and/or status alert through a network such as the Medtronic CareLink® Network developed by Medtronic, Inc., of Minneapolis, Minn., or some other network linking patient 14 to a clinician. In this manner, a computing device or user interface of the network may be the external computing device that delivers an HF score and/or status alert to a user.

In some examples, one or more steps in the generation of the HF risk score may occur within a device external of patient 14, e.g., within external device 24 or external device 120. In this manner, IMD 16 may detect and store patient parameters before transmitting the patient parameters to a different computing device. IMD 16 may spontaneously transmit the diagnostic information to the network or in response to an interrogation request from a user.

The patient data identified and/or generated and stored by IMD 16 may include one or more of the patient parameters described herein. Each of these patient parameters may be used to generate a diagnostic parameter that may be used in the algorithm to determine HF status when the value of the diagnostic parameter meets a respective threshold. Example patient parameters that IMD 16 may detect may include oversensing episodes (e.g., electromagnetic interference, lead failure, T-wave oversensing), shock events, atrial fibrillation, ventricular tachycardia (VT), ventricular fibrillation (VF), junctional rhythms, supraventricular tachycardia (SVT) (e.g., sinus tachycardia or atrial tachycardia), monomorphic VT, anti-tachycardia pacing (ATP) events, non-sustained VT or VF events, and a mode-switch episode (e.g., changing from a tracking mode to a non-tracking mode at the onset of a pathological rhythm such as atrial fibrillation or atrial tachycardia). Other patient parameters may include any sensed or detected parameters related to cardiac rhythms, electrical or hemodynamic cardiac episodes, patient activity, a nighttime heart rate, a difference between night and day heart rate, a heart rate variability, patient posture, coughing, intensity and/or frequency of coughing, congestion of patient, or any other physiological event or condition of the patient. In other examples, other patient parameters may be stored that may be useful in the detection of HF risk, e.g., blood pressure, right ventricular pressure, pulmonary artery pressure, patient temperature, lung volume, lung tidal volume, lung density, breathing rate or even biomarkers such as a brain natriuretic peptide (BNP), troponin, or related surrogates. These parameters may be detected or sensed via one or more electrodes or sensors in communication with IMD 16 (e.g., wired or wireless sensors).

As will be described in greater detail below, one or more external devices (and/or IMD 16 in some cases) may determine when the algorithm falsely identified an HF event or conversely when the algorithm failed to identify an HF event. For the case when the algorithm falsely identified an HF event (i.e., false positive), parameter thresholds may be raised such that false alerts would not have occurred (i.e., adjust the algorithm based on previous HF episodes). For the case when the HF-triage algorithm failed to identify HF episode(s) (i.e., false negative), compare the values of the contributing parameters with the parameter values of previous true positive events and adjust the parameter threshold that did not trigger a HF status.

FIG. 4 is a block diagram illustrating an example system 140 that includes an external device, such as a server 120, and one or more computing devices 146A-146N (collectively, “computing devices 146”), that are coupled to the IMD 16 and external device 24 shown in FIG. 1 via a network 144. Network 144 may be generally used to transmit clinician input, patient data, diagnostic parameters, patient management reports, or any other information between any devices of system 140. Network 144 may allow the clinician to monitor and/or treat patients remote from the clinician. In this example, IMD 16 may use its communication circuitry 88 to communicate with external device 24 via a first wireless connection, and to communication with an access point 142 via a second wireless connection, which may be the same type or different types of wireless connections. In the example of FIG. 4, access point 142, external device 24, server 120, and computing devices 146 are interconnected, and able to communicate with each other, through network 144. In some cases, one or more of access point 142, external device 24, server 120, and computing devices 146 may be coupled to network 144 through one or more wired or wireless connections. IMD 16, external device 24, server 120, and computing devices 146 may each comprise one or more processors, such as one or more microprocessors, DSPs, ASICs, FPGAs, programmable logic circuitry, or the like, that may perform various functions and operations, such as those described herein.

Access point 142 may comprise a device that connects to network 144 via any of a variety of connections, such as telephone dial-up, digital subscriber line (DSL), or cable modem connections. In other examples, access point 142 may be coupled to network 144 through different forms of connections, including wired or wireless connections. In some examples, access point 142 may be co-located with patient 14 and may comprise one or more programming units and/or computing devices (e.g., one or more portable or home monitoring units) that may perform various functions and operations described herein. For example, access point 142 may include a home-monitoring unit that is co-located with patient 14 and that may monitor the activity of IMD 16 (e.g., receive information or data from IMD 16) and/or receive patient data used by server 120 to generate diagnostic parameters. In some examples, server 120 or computing devices 146 may control or perform any of the various functions or operations described herein, e.g., apply a HF monitoring algorithm, including comparing diagnostic parameters to respective thresholds, and identify false determinations by the algorithm, and modify the algorithm, e.g., various thresholds of the algorithm, based on the identified false determinations.

Computing devices 146 may include tablet computers, workstations, notebook computers, mobile phones, personal digital assistants, or any other computing device that may be used by a healthcare professional to monitor and/or treat a patient. For example, computing device 146A may be carried by a clinician tasked with monitoring the health of one or more patients. Computing device 146A, or any other computing device 146, may present the patient management report as a centralized information source about the patients that the clinician is tracking. The clinician may use the patient management report as a way to triage patients without separately reviewing data from each patient periodically. The patient management report may be delivered as a web page through a web browser, within a specific software application, or in any other digital format. In other examples, the clinician may receive the patient management report as a printed report instead of an interactive or non-interactive digital report via computing device 146A.

The patient management report may be generated and delivered periodically. For example, server 120 may generate the patient management report on a daily basis at a time requested by the clinician and deliver the report to one or more computing devices 146. In other examples, the patient management report may be delivered to a clinician account managed by server 120. When the clinician logs into the clinician account from any computing device 146 or external device 24, server 120 may deliver the patient management report or notify the clinician that a new patient management report is available. The periodic delivery of patient management reports may be performed daily or at some other predetermined interval set by a clinic, the clinician, a manufacturer of IMD 16, or an account manager of server 120. In other examples, patient management reports may be generated in response to a request from the clinician or in response to one or more new diagnostic parameters that may indicate urgent attention by the clinician is necessary.

Customization of the patient management reports may be updated by a clinician from one or more devices (e.g., computing devices 146 or external device 24) that have access to server 120 via network 144. Server 120 may maintain a clinician account for each clinician associated with server 120. Anytime server 120 receives clinician input identifying an updated preference or set of preferences, server 120 may update the appropriate preferences in the clinician account. These preferences received from the clinician may include selected reporting characteristics, patient management report delivery timing, which diagnostic parameters to include, or even when to hide certain diagnostic parameters from the patient management report (e.g., only including diagnostic parameters not previously presented in a patient management report to the clinician such that continual patient conditions do not bury new issues from one or more patients).

In some cases, a repository 134 (FIG. 5) accessible by server 120 may be configured to provide a secure storage site for archival of patient information (e.g., patient history 136 or IMD data 138, illustrated in FIG. 5) that has been collected from IMD 16, external device 24, computing devices 146, and/or any other device. Network 144 may comprise a local area network, wide area network, or global network, such as the Internet. In some cases, external device 24 or server 120 may generate the patient management reports in web pages or other documents for viewing by and trained professionals, such as clinicians, via viewing terminals associated with computing devices 146. The system of FIG. 4 may be implemented, in some aspects, with general network technology and functionality similar to that provided by the Medtronic CareLink® Network developed by Medtronic, Inc., of Minneapolis, Minn.

In the illustrated example of FIG. 4, system 140 also includes at least one electronic medical records (EMR) system 147 accessible via network 144. EMR system 147 stores a variety of data pertaining to medical records of patient 14. The data stored by EMR system 147 may not otherwise be available to server 120, e.g., may not be retrieved from IMD 16 and/or external device 24, and may not pertain to the operation of the IMD or treatment of patient 12 with the IMD. The data stored by EMR system 147 may include, as examples, data pertaining to medical symptoms and events experienced by patient 14, treatments provided to patient 14, and hospitalizations or other clinical visits of patient 14, include dates, times, and durations of these. In some examples, the data stored by EMR system 147 include information that indicates whether patient 14 experiences an HFH or other HF event, including the dates, time, and durations of such events. Data from EMR system 147 retrieved by server 120 may be stored in repository 134 as patient history 136. Server 120 may retrieve data from EMR system 147 on a periodic basis or in response to another event, such as determination of an HF score and/or status for patient 14.

FIG. 5 is a functional block diagram illustrating an example configuration of an external device, such as a remote server 120, configured to apply an HF monitoring algorithm to diagnostic data for one or more patients. FIG. 5 illustrates only one particular example configurations of server 120, and many other example configurations of server 120 may be used in other instances. For example, server 120 may include additional components and run multiple different applications, including different algorithms.

As shown in the specific example of FIG. 5, sever 120 may include and/or house processing circuitry 122, memory 124, and at least one network interface 126, user interface 128, and power source 132. Server 120 may be in communication with repository 134, such that repository 134 is located external of server 120. In other examples, repository 134 may include one or more storage devices within an enclosure of server 120. Server 120 may also include an operating system, which may include modules and/or applications that are executable by processing circuitry 122 and server 120. Each of components 122, 124, 126, 128, 132 and 134 may be interconnected (physically, communicatively, and/or operatively) for inter-component interaction.

Processing circuitry 122, in one example, is configured to implement functionality and/or process instructions for execution within server 120, such as generating diagnostic data and running algorithms for determining an HF score and/or status based on the diagnostic data, as described herein. Various instructions and data, e.g., threshold values, used by processing circuitry 122 when executing the algorithm may be stored in memory 124 in an algorithm module 125. Processing circuitry 122 may comprise, as examples, one or more microprocessors, DSPs, or any other processing circuitry described herein.

Memory 124 may generally store instructions that, when executed by processing circuitry 122, causes the processing circuitry and server 120 to provide functionality, e.g., as described herein. Memory 124, in some examples, is described as a computer-readable storage medium, e.g., a non-transitory computer-readable storage medium. Memory 124 may include any volatile or non-volatile memory described herein.

Repository 134, in some examples, also includes one or more computer-readable storage media, and may include any volatile or non-volatile memory described herein. Repository 134 may be configured to store larger amounts of information than memory 124. Repository 134 may further be configured for long-term storage of information. Repository 134 may store information in any of a variety of database or other data organization structures. Repository 134 may be configured to store patient information and/or patient data used to generate diagnostic reports and adjust algorithms. For example, repository 134 may store patient history 136 and IMD data 138. Patient history 136 may include any history, information, observations, or any other data related to the monitoring, diagnosis, running of algorithms, and/or treatment of one or more patients. IMD data 138 may include sensed or detected patient data obtained from one or more implantable medical device such as IMD 16, including any of the patient or diagnostic parameter data described herein. IMD data 138 may include data from one or more patients. For each of patient history 136 and IMD data 138, repository 134 may maintain separate files for each patient or otherwise maintain security of the data to retain patient privacy.

Server 120, in some examples, also includes a network interface 126. Server 120, in one example, utilizes network interface 126 to communicate with other computing devices, external devices (e.g., external device 24), medical devices (e.g., IMD 16), systems (such as EMR system 147), or more networks, such as network 144 in FIG. 4. Network interface 126 may be a network interface card, such as an Ethernet card or other wired interface. In other examples, network interface 126 may include an optical transceiver, a radio frequency transceiver, or any other type of device that can send and receive information. Other examples of such network interfaces may include Bluetooth, 3G and WiFi radios in mobile computing devices as well as USB. In some examples, server 120 utilizes network interface 126 to wirelessly communicate with another computing device (e.g., computing device 146N of FIG. 4 or other networked computing device.

Server 120, in one example, also includes one or more user interfaces 128. User interface 128 may include a touch-sensitive and/or a presence-sensitive screen, mouse, a keyboard, a voice responsive system, camera, or any other type of device for detecting a command from a user. In one example, user interface 128 may include a touch-sensitive screen, sound card, a video graphics adapter card, or any other type of device for converting a signal into an appropriate form understandable to humans or machines. In addition, user interface 128 may include a speaker, a cathode ray tube (CRT) monitor, a liquid crystal display (LCD), or any other type of device that can generate intelligible output to a user. User interface 128 may be, but is not necessarily, co-located with other components of server 120. In some examples, one or more computing devices 146, external devices 24, or other computing devices, e.g., a tablet computing device or a personal computer used by the clinician, are able to access server 120 via network 144 (FIG. 4) to provide an interface for a user to server 120.

Server 120, in some examples, includes one or more power sources 132, which provide power to server 120. Generally, power source 132 may utilize power obtained from a wall receptacle or other alternating current source. However, in other examples, power source 132, may include one or more rechargeable or non-rechargeable batteries (e.g., constructed from nickel-cadmium, lithium-ion, or other suitable material). In other examples, power source 132 may be a power source capable of providing stored power or voltage from another power source.

As described herein, processing circuitry, e.g., processing circuitry 122 of server 120, may adjust an algorithm for determining an HF score and/or status based on identifying one or more false determinations of the score or status made by the algorithm. Generally, the current parameters of the algorithm, e.g., thresholds and other values, as well as adjustments to the algorithm, may be stored in algorithm module 125. However, in some examples, one or more aspects of updating and adjusting the algorithm may be performed by a different device, such as external device 24 or another external computing device.

As described herein, processing circuitry 122 may receive parameter data for at least one patient. The patient data may include sensed or detected parameters from IMD 16 for each patient, patient medical history information, e.g., from EMR system 147, or any other information that may be used to generate each of the diagnostic parameters. The patient data may be received from one source (e.g., IMD 16) or multiple sources (e.g., IMD 16, external device 24, repository 134, and/or any other local or remote database of the patients). Server 120 may store any received patient data as patient history 136 and/or IMD data 138 within repository 134. Processing circuitry 122 may then retrieve the patient data as needed from repository 134, e.g., for evaluation of the data according to an HF monitoring algorithm.

According to an HF monitoring algorithm, processing circuitry 122 may also determine a value for at least a subset of the diagnostic parameters based on the received patient data. In some examples, the value of one or more diagnostic parameters may have been determined by another device, such as IMD 16 and/or external device 24. Processing circuitry 122 may then compare the diagnostic parameter values to one or more respective thresholds of each diagnostic parameter. In example implementations of a HF monitoring algorithm, diagnostic parameters meeting their threshold may contribute to a score, which may represent an evidence level or probability of HFH or another HF event. In some examples, the score may also be compared to one or more thresholds to determine a status. The status may be binary, such as concerned or not concerned, or some other characterization of the score as being in one of a plurality of levels of concern or probability, such as low, medium, and high.

Server 120 may transmit the score, status, and/or underlying patient parameter data to a clinician or other user, e.g., in the form of a report. Network interface 126 may be configured to transmit the information to a computing device, e.g., external device 24 or computing device 146 of FIG. 4 for review by the user. The computing device may present the patient management report via a user interface.

The diagnostic parameters may be generated using a sensed component (e.g., a value from IMD 16 or another medical device) and/or a patient information component entered by a clinician. In this manner, at least one diagnostic parameter may provide a synthesis of sensed data and characteristics of the patient to synthesize useful information about any changes to the patient condition and/or treatment and whether to adjust the algorithm. One or more diagnostic parameters may thus provide additional information than would otherwise be available from only IMD 16 or only clinician notes. For example, patient history 136 may include clinician entered information and/or observations about the patient not sensed or detected by a medical device. IMD data 138 may include patient data sensed or detected from one or more sensors, which may be included in IMD 16 and/or other medical devices.

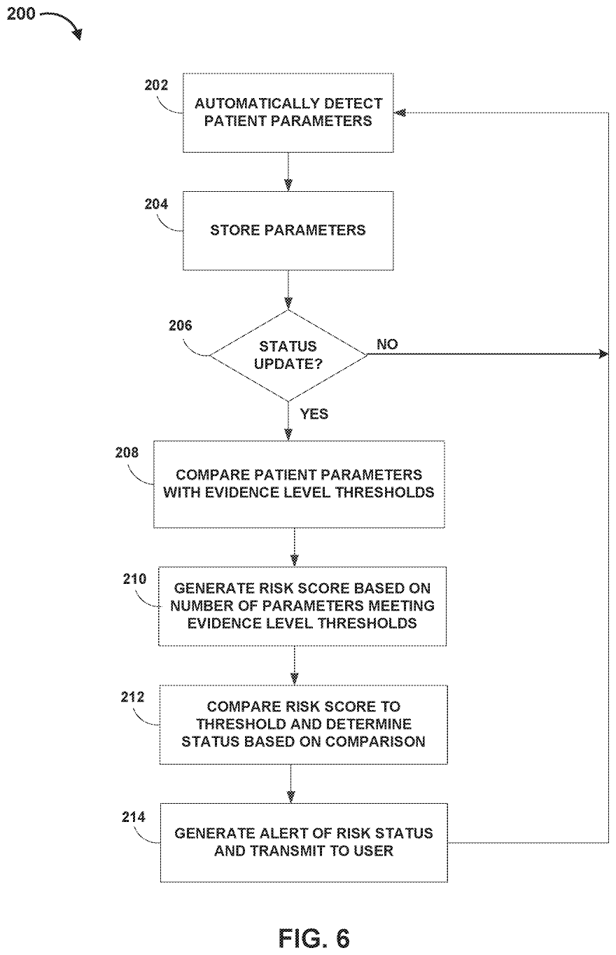

FIG. 6 is a flow diagram of an example technique for implementing an algorithm to determine a HF score and/or status based on patient diagnostic data. FIG. 6 will be described with IMD 16 detecting patient parameters and sever 120 generating HF risk scores for the patient, but other examples of the same technique may be implemented by one or more other devices, e.g., IMD 16, external device 24, and/or external device (such as a server) 120 based on diagnostic data received from IMD 16 or one or more other medical devices.

More particularly, FIG. 6 illustrates method 200 in which IMD 16 automatically detects the patient parameters from various electrodes, sensors, and therapy information (202). IMD 16, e.g., processing circuitry 80 of IMD 16, then stores the patient parameters in patient parameter data 85 of memory 82 (204). Server 120 and/or IMD 16 may determine whether it is time to update an HF status of patient 14, e.g., according to a schedule or user request (206). In some examples, server 120 may retrieve patient diagnostic parameter data from IMD 16 and update the patient HF status on a periodic, e.g., daily, basis. If it is not time for a transmission and/or status update (“NO” branch of block 206), IMD continues to detect patient parameters (202).

In some examples, the period, or evaluation window, for automatic transmissions and HF status updates may be other than daily, such as every three months, or every thirty days or seven days, for example. In some examples, the transmission and/or HF status update may occur as frequently as every hour or as infrequently as several months. In some examples, the patient diagnostic data transmission and/or HF status update may be device initiated, such as in response to an increased thoracic impedance or other fluid parameter value.

If it is time for a diagnostic parameter transmission and HF status update (“Yes” branch of block 206), processing circuitry, e.g., processing circuitry 122 of server 120, compares each of the patient parameters with the evidence level threshold of that parameter (208). Once the comparisons are complete, processing circuitry 80 may generate a risk score based on the number of parameters meeting evidence level thresholds (210). The risk score may then be compared to a risk level threshold and the HF status is determined based on the comparison (212). For example, no parameters meeting their thresholds may be a “low risk” status, one parameter meeting its threshold may be a “medium risk” status, and two or more parameters meeting their thresholds may be a “high risk” status. In other examples, the risk status may be generated as a fraction, percentage, or other value that represents the number of parameters meeting their thresholds. In some examples, processing circuitry 122 may generate the risk statuses with a statistical analysis of the patient parameter values instead of using the number of parameters meeting a threshold.

After the risk status is determined, processing circuitry 122 generates an alert of the risk status and transmits the alert to the user via network interface 126, e.g., via network 144 and computing device 146 (214). As described herein, the alert may be transmitted on a schedule or as soon as communication is possible to another device or access point. In some examples, the HF risk status may only be transmitted when requested by a user. In some examples, the alert may also include more detailed information regarding the patient parameters included in the risk status.

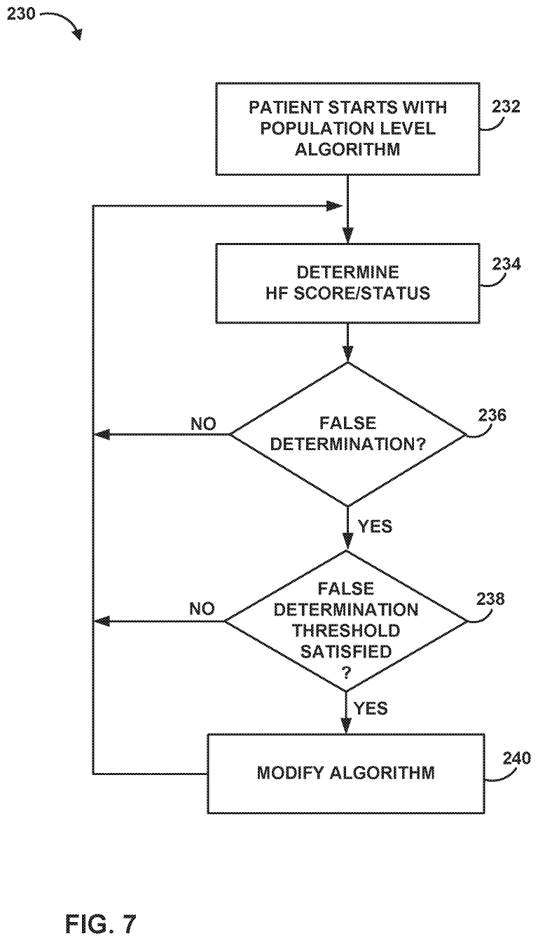

FIG. 7 is a flow diagram of an example technique for monitoring and adjusting an algorithm for determining an HF score and/or status. FIG. 7 will be described with respect to an example in which server 120 monitors the performance of the algorithm and adjusts the algorithm, if necessary, but other examples of the same technique may be implemented by one or more other devices (e.g., IMD 16 and/or external device 24.

More particularly, FIG. 7 illustrates method 230 in which server 120 initially applies a population-level algorithm to patient diagnostic data of patient 14 (232). Server 120 determines an HF score and/or status based on the application of the patient diagnostic data to the algorithm, e.g., as described herein, such as with respect to FIG. 6. A population-level algorithm may refer to an algorithm developed for HF patients with average clinical characteristics. For example, the various evidence level thresholds and the risk level threshold may be set at values determined based on one or more clinical studies of patient parameter data and outcomes from a population of patients having average characteristics,

However, HF patients can be complex and fall on a continuum. While some patients may be very sick and only be active for an average of one hour per day, other HF patients may be relatively healthier and be active, e.g., two to three times more active than very sick patients (e.g., active for 2-3 hours per day). Using the same algorithm, e.g., thresholds, uniformly for all HF patients can be a starting point. As more patient data and information is collected, the algorithm can be adjusted on a per patient basis to customize the algorithm for a given patient with the primary goal of lowering the false alert rate, while preserving the sensitivity (i.e., detection of a true HF event). In some examples, the primary goal may be the reduction of one of false positives and false negatives.

Processing circuitry, e.g., processing circuitry 122 of server 120, determines whether the application of the algorithm to the patient diagnostic data resulted in a false determination of the HF score and/or status for patient 14 (236). A false determination may be either a false positive determination, e.g., indication of an impending HF event when such an event did not occur, or a false negative indication, e.g., failure to indicate an HF event that did in fact occur. Processing circuitry 122 may receive data indicating whether or not an HF event, e.g., HFH, actually occurred. The data may indicate whether the event occurred within a threshold time period from the determination by the algorithm. The data may be entered by a user, e.g., via external device 24 or computing device 146. In some examples, processing circuitry 122 may retrieve the data indicating whether an HF event occurred from an EMR system 147 via network 144. EMR system 147 may store clinical data for patient 14 indicating whether patient 14 experienced an HF event and the time, e.g., date, or such occurrences. If the algorithm indicated that an HF event occurred and the EMR data indicates that no HF event did occur, then there is a false determination (236), a false positive. On the other hand, if the HF risk status did not indicate an HF event when an HF event did occur, then there is a false determination (236), a false negative.

If there is no false determination (NO from block 236), then method 230 returns to block 234 and continues to apply the algorithm without modification. If there is a false determination (YES from block 236), then method 230 proceeds to block 238 to determine whether the false determination threshold or other false determination criterion (238) has been satisfied. In some examples, processing circuitry 122 may take an action, such as modifying the HF monitoring algorithm for patient 14, based on the occurrence of a single false determination. In other examples, such as that illustrated by example method 230, processing circuitry 122 takes action when a false determination threshold or other criterion is satisfied.

Depending on the number of false determinations, the false determination threshold (also referred to as a criterion) may have been satisfied (238). If the false determination threshold or criterion has not been satisfied (“NO” from block 238), then method 230 returns to block 234 and continues to apply the algorithm without modification. If the false determination threshold or criterion has been satisfied (“YES” from block 238), then processing circuitry 122 modifies the HF monitoring algorithm (240). In some examples, false positive and negative determinations are separately counted, and numbers of the respective false determinations are compared to respective false determination thresholds. In some examples, the false determination threshold or criterion is a threshold number of false determinations occurring within a threshold period of time. As described herein, modification of the algorithm may include modification of one or more of the thresholds used by the algorithm to determine the HF score and/or status. Whether or not the algorithm has been modified, processing circuitry 122 may continue to apply the algorithm to patient diagnostic data, e.g., newly received patient diagnostic data (234).

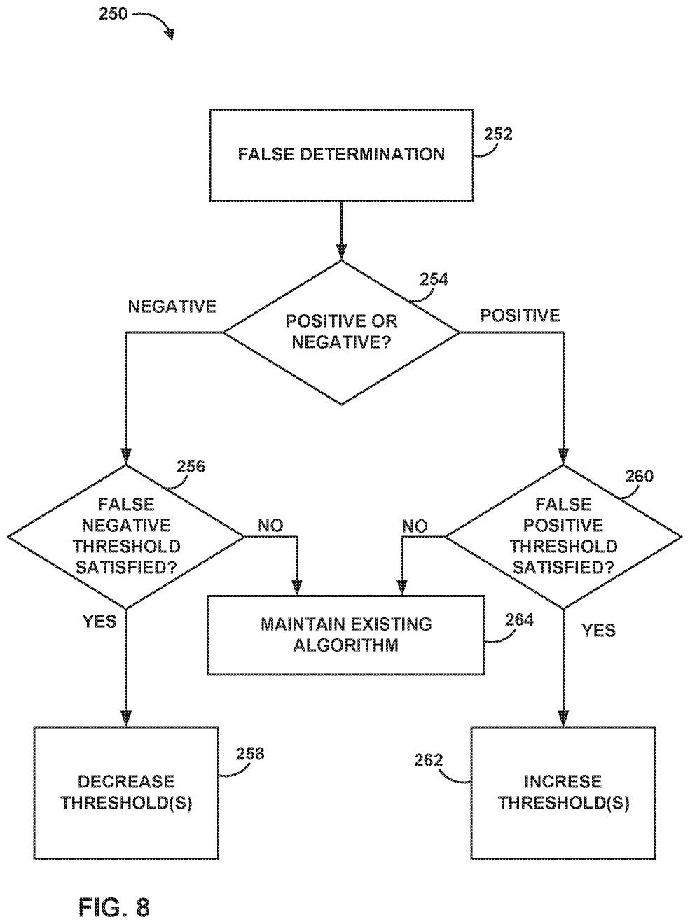

FIG. 8 is a flow diagram of an example technique for monitoring and adjusting an algorithm for determining a HF score and/or status based on identification of negative and positive false determinations. Although described in the context of an example in which processing circuitry 122 of server 120 performs method 250, the example method may be additionally or alternatively performed by processing circuitry of one or more other devices, such as IMD 16 or external device 24, in other examples. Method 250 may be an example implementation of method 230. For example, a positive determination of the occurrence of a false determination (“YES” of block 236 from FIG. 7) may result in a false determination (252) of the example of FIG. 8.

According to the example method 250, after processing circuitry 122 ascertains that a false determination has been made (252), the processing circuitry determines whether the false determination was positive or negative (254). If processing circuitry 122 ascertains the false determination was a false negative, the processing circuitry proceeds to determine whether the false negative threshold or criterion was satisfied (256). If the false negative threshold or criterion is not satisfied (“NO” branch of block 256), the existing algorithm is maintained (264). If the false negative threshold is satisfied (“YES” branch of block 256), processing circuitry 122 can decrease one or more thresholds in the existing algorithm (258). In some examples, processing circuitry 122 decreases the evidence level threshold for one or more of the diagnostic parameters, e.g., the threshold which a value of the diagnostic parameter must satisfy to count as evidence of an impending HF event according to the HF monitoring algorithm.

Similar to the determination of a false negative, if processing circuitry 122 ascertains the false determination was a false positive, the processing circuitry determines whether the false positive threshold or criterion was satisfied (260). If the false positive threshold or criterion is not satisfied (“NO” branch of block 260), the existing algorithm is maintained (264). If the false positive threshold or criterion is satisfied (“YES” branch of block 260), processing circuitry 122 can increase one or more thresholds in the existing algorithm (262). In some examples, processing circuitry 122 increases a risk level threshold, e.g., the threshold which a value risk score must satisfy to result in an alert of a particular HF event status.

The false positive and false negative thresholds or criteria may allow for “X” number of successive false alerts (“X” is programmable and can be 2 or 3 false alerts but could also be one false alert or a higher number of false alerts) in a specific duration (e.g., 6 months). The false positive and false negative thresholds or criteria may be the same or different. For example, the false positive and false negative thresholds or criteria may both allow for 2 false alerts in 6 months. In another example, the false positive threshold or criterion may allow for 2 false alerts in 8 months, and the false negative threshold or criterion may allow for 3 false alerts in 4 months.

In some applications, reducing one of false negatives or false positives may be of greater importance than the other. In such applications, in contrast to the example shown in FIG. 8, the determination of whether the less important of false positive or false negative threshold has been satisfied may occur only after the more important threshold has been satisfied and a corresponding modification of the algorithm made. For an example where reducing false negatives is given greater emphasis, a false negative threshold must be first satisfied (“YES” of block 256) before determining whether a false positive threshold is satisfied (260). In other examples, reducing false positives may be given greater emphasis. There may be various application reasons why detecting one false determination over another is important. For example, clinicians could be interrupted too often with false positive alerts and the algorithm is used to focus on help reducing the time burden of patients on clinicians. Withholding modifications based on the less important category of false determinations may occur in a variety of ways, including not identifying the less important false determinations, not comparing a number of the less important false determinations to a threshold, and/or not modifying the algorithm based on the less important false determinations until a modification of the algorithm based on the more important category of false determination.

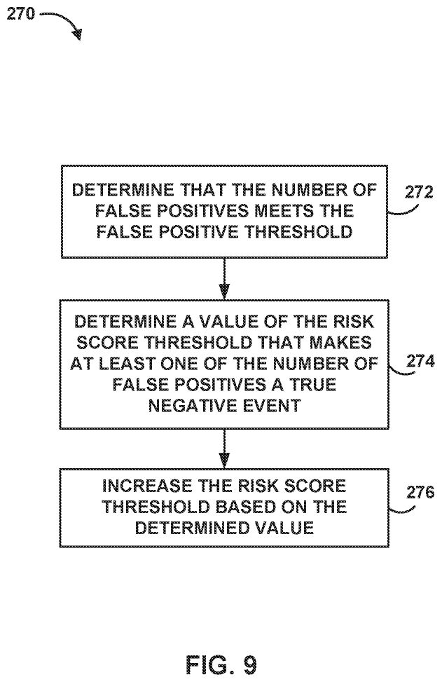

FIG. 9 is a flow diagram of an example technique for monitoring and adjusting an algorithm for determining a HF score and/or status in response to false positives. Although described in the context of an example in which processing circuitry 122 of server 120 performs method 270, the example method may be additionally or alternatively performed by processing circuitry of one or more other devices, such as IMD 16 or external device 24, in other examples. In general, method 270 of FIG. 9 may represent an example implementation of method 250 of FIG. 8 when the false positive threshold or criterion is satisfied (“YES” of block 260 of FIG. 8).

According to the example of method 270, processing circuitry 122 determines that the number of false positives meets the false positive threshold (272). In the illustrated example, processing circuitry 122 modifies the algorithm according to each patient's data and information by adjusting one or more thresholds in response to the determination. In particular, processing circuitry 122 determines a value of the risk score threshold that would result in at least one of the false positive determinations by the algorithm being reclassified as a true negative (274). As described herein, in order to apply the algorithm to the values of the diagnostic parameters of the patient, processing circuitry 122 can determine an evidence level for each of the diagnostic parameters based on the respective value for the diagnostic parameter, determine a risk score based on the plurality of evidence levels, compare the risk score to a risk score threshold, and determine the heart failure risk status based on the comparison of the risk score to the risk score threshold. Processing circuitry 122 may then modify the algorithm to increase the risk score threshold for patient 14 based on the determined value (276). Processing circuitry 122 may increase the risk score threshold to the value that would have changed a single false positive to a true negative, the lowest value that would have changed one of a plurality of false positives to a true negative, the value that would have changed each of a plurality of false positives to true negatives, a mean or median of values that would have changed each of a plurality of false positives to true negatives, or some other updated threshold level based on one or more values that would have changed one or more false positives to false negatives.

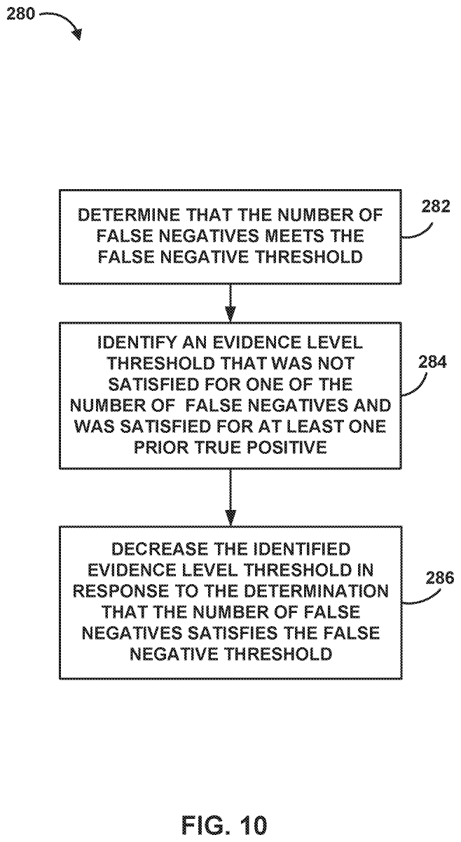

FIG. 10 is a flow diagram of an example technique for monitoring and adjusting an algorithm for determining a HF score and/or status in response to false negatives. Although described in the context of an example in which processing circuitry 122 of server 120 performs method 280, the example method may be additionally or alternatively performed by processing circuitry of one or more other devices, such as IMD 16 or external device 24, in other examples. In general, method 280 of FIG. 10 may represent an example implementation of method 250 of FIG. 8 when the false negative threshold or criterion is satisfied (“YES” of block 256 of FIG. 8).

Method 270 of FIG. 9 may be similar to method 280 of FIG. 10 regarding the features of the algorithm for determining the HF score and/or status. For method 280, processing circuitry 122 will determine whether the number of false negatives meets the false negative threshold (282). If the number of false negatives meet the false negative threshold, processing circuitry 122 may identify an evidence level threshold that was not satisfied for one of the number of false negatives and was satisfied for at least one prior true positive (284). Processing circuitry 122 may then decrease the identified evidence level threshold in response to the determination that the number of false negatives satisfies the false negative threshold (286). The adjusted algorithm may then continue to monitor for false determinations.

The examples methods of FIGS. 7-10 include modification of an algorithm for determining a HF score and/or status in response to identifying false determinations by the algorithm. The techniques described herein also include not modifying the algorithm in response to identifying true determinations by the algorithm. In some examples, processing circuitry determines that a number of true determinations by the algorithm satisfies, e.g., is greater than or equal to, a true determination threshold, and leaves the algorithm unmodified in response to the determination. In some examples, the processing circuitry may reduce or zero a false determination count in response to determining that the number of true determinations satisfies the true determination threshold.

The disclosure also contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

The techniques described in this disclosure, including those attributed to IMD 16, external device 24, and server 120 and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. For example, any of the techniques or processes described herein may be performed within one device or at least partially distributed amongst two or more devices, such as between IMD 16, external device 24, and server 120. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media.

Various examples have been described. These and other examples are within the scope of the following claims.

1: A system comprising:
one or more medical devices configured to, for each of a plurality of periods, determine a respective value for each of a plurality of parameters of a patient; and
processing circuitry configured to:
for each of the plurality of periods, determine at least one of a heart failure risk score or status for the patient based on the determined values for the period using one or more thresholds;
determine a number of the determined heart failure risk scores or statuses that were false determinations;
determine that the number of false determinations for the patient satisfies a false determination criterion; and
modify at least one of the one or more thresholds for the patient in response to the determination that the number of false determinations for the patient satisfies the false determination criterion.

2: The system of claim 1, wherein, in order to determine the at least one of the heart failure risk score or status, the processing circuitry is configured to:
determine an evidence level for each of the parameters based on the respective value for the parameter;
determine a risk level based on the plurality of evidence levels; compare the risk level to a risk level threshold; and
determine the heart failure risk status based on the comparison of the risk level to the risk level threshold.

3: The system of claim 2, wherein the processing circuitry is configured to adjust the risk level threshold in response to the determination that the number of false determinations for the patient satisfies the false determination criterion.

4: The system of claim 3, wherein the false determination criterion comprises a false positive criterion, and the processing circuitry is configured to:
determine a number of the determinations for the patient that were false positives;
determine that the number of false positives satisfies the false positive criterion; and
increase the risk level threshold in response to the determination that the number of false positives satisfies the false positive criterion.

5: The system of claim 4, wherein the processing circuitry is configured to:
determine a value of the risk level threshold that makes at least one of the number of false positives a true negative; and
increase the risk level threshold based on the determined value.

6: The system of claim 2, wherein, in order to determine the at least one of the heart failure risk score or status, the processing circuitry is configured to:
apply a respective one or more evidence level thresholds to each of the plurality of parameter values; and
determine the evidence level for each of the parameters based on the comparison,
wherein the processing circuitry is further configured to adjust at least one of the evidence level thresholds in response to the determination that the number of false determinations for the patient satisfies the false determination criterion.

7: The system of claim 6, wherein the false determination criterion comprises a false negative criterion, and the processing circuitry is configured to:
determine a number of the determinations for the patient that were false negatives;
determine that the number of false negatives satisfies the false negative criterion; and
decrease the evidence level threshold in response to the determination that the number of false negatives satisfies the false negative criterion.

8: The system of claim 7, wherein the processing circuitry is configured to:
identify an evidence level threshold that was not satisfied for one of the number of false negatives and was satisfied for at least one prior true positive; and
decrease the identified evidence level threshold in response to the determination that the number of false negatives satisfies the false negative criterion.

9: The system of claim 1, wherein the processing circuitry comprises processing circuitry of at least one of the one or more medical devices or a remote system in communication with at least one of the medical devices.

10: The system of claim 1, wherein the false determination criterion comprises a threshold number of false determinations during a period of time.

11: The system of claim 1, further comprising a user interface, wherein the false determination criterion is programmable by a user via the user interface.

12: The system of claim 1, wherein the processing circuitry is configured to determine the number of the determinations that were false based on at least one of user input or data from an electronic medical record system indicating whether the patient experienced a heart failure event.

13: The system of claim 1, wherein the processing circuitry is further configured to:
determine a number of the determinations that were correct;
determine that the number of correct determinations for the patient satisfies a correct determination criterion; and
maintain the algorithm unmodified for the patient based on the determination that the number of false determinations for the patient satisfies the correct determination criterion.

14: The system of claim 13, wherein the processing circuitry is further configured to modify at least one of determined number of false determinations or the false determination criterion based on the determination that the number of false determinations for the patient satisfies the correct determination criterion.

15: The system of claim 1, wherein the processing circuitry is configured to automatically modify the at least one of the one or more thresholds in response to the determination.

16: The system of claim 1, wherein the determination that the number of false determinations satisfies the false determination criterion comprises a determinization that the number of false determinations is at least equal to the false determination criterion.

17: A system comprising:
one or more medical devices configured to, for each of a plurality of periods, determine a respective value for each of a plurality of parameters of a patient; and
processing circuitry configured to:
for each of the plurality of periods, determine at least one of a heart failure risk score or status for the patient based on the determined values for the period using one or more thresholds;
determine a number of the determined heart failure risk scores or statuses that were false determinations;
determine that the number of false determinations for the patient satisfies a false determination criterion; and
automatically modify at least one of the one or more thresholds for the patient in response to the determination that the number of false determinations for the patient satisfies the false determination criterion.

18: A method comprising:
for each of a plurality of periods, determining a respective value for each of a plurality of parameters of a patient with one or more medical devices;
for each of the plurality of periods, determining at least one of a heart failure risk score or status for the patient based on the determined values for the period using one or more thresholds;
determining a number of the determined heart failure risk scores or statuses for the patient that were false determinations;
determining that that the number of false determinations for the patient satisfies a false determination criterion; and
modifying at least one of the one or more thresholds for the patient in response to the determination that the number of false determinations for the patient satisfies the false determination criterion.

19: The method of claim 18, wherein determining the at least one of the heart failure risk score or status comprises:
determining an evidence level for each of the parameters based on the respective value for the parameter;
determining a risk level based on the plurality of evidence levels;
comparing the risk level to a risk level threshold; and
determining the heart failure risk score or status based on the comparison of the risk level to the risk level threshold.

20: The method of claim 19, wherein adjusting one or more thresholds comprises adjusting the risk level threshold in response to the determination that the number of false determinations for the patient satisfies the false determination criterion.

21: The method of claim 20, wherein the false determination criterion comprises a false positive criterion, the method further comprising:
determining a number of the determinations for the patient that were false positives; and
determining that the number of false positives satisfies the false positive criterion,
wherein adjusting the risk level threshold comprises increasing the risk level threshold in response to the determination that the number of false positives satisfies the false positive criterion.

22: The method of claim 21, wherein increasing the risk level threshold comprises:
determining a value of the risk level threshold that makes at least one of the number of false positives a true negative; and
increasing the risk level threshold based on the determined value.

23: The method of claim 19, wherein determining the at least one of the heart failure risk score or status comprises:
applying a respective one or more evidence level thresholds to each of the plurality of parameter values; and
determining the evidence level for each of the parameters based on the comparison,
wherein adjusting one or more thresholds comprises adjusting at least one of the evidence level thresholds in response to the determination that the number of false determinations for the patient satisfies the false determination criterion.

24: The method of claim 23, wherein the false determination criterion comprises a false negative criterion, the method further comprising:
determining a number of the determined heart failure risk scores or statuses that were false negatives; and
determining that the number of false negatives satisfies the false negative criterion,
wherein adjusting at least one of the evidence level thresholds comprises decreasing the evidence level threshold in response to the determination that the number of false negatives satisfies the false negative criterion.

25: The method of claim 24, wherein decreasing the evidence level threshold comprises:
identifying an evidence level threshold that was not satisfied for one of the number of false negatives and was satisfied for at least one prior true positive; and
decreasing the identified evidence level threshold in response to the determination that the number of false negatives satisfies the false negative criterion.

26: The method of claim 18, wherein the false determination criterion comprises a threshold number of false determinations during a period of time.

27: The method of claim 18, further comprising receiving user input programming the false determination criterion via a user interface.

28: The method of claim 18, further comprising receiving at least one of user input or data from an electronic medical record system indicating whether the patient experienced a heart failure event, wherein determining the number of the determinations for the patient that were false comprises determining the number of the determination that were false based on the at least one of user input or data.

29: The method of claim 18, further comprising:
determining a number of the determinations that were correct;
determining that the number of correct determinations for the patient satisfies a correct determination criterion; and
maintaining the algorithm unmodified for the patient based on the determination that the number of false determinations for the patient satisfies the correct determination criterion.

30: The method of claim 29, further comprising modifying at least one of determined number of false determinations or the false determination criterion based on the determination that the number of false determinations for the patient satisfies the correct determination criterion.

31: The method of claim 18, wherein modifying the at least one of the one or more thresholds comprises automatically modifying the at least one of the one or more thresholds by processing circuitry of a medical device system.

32: The method of claim 31, wherein the determination that the number of false determinations satisfies the false determination criterion comprises a determinization that the number of false determinations is at least equal to the false determination criterion.

33: A computer-readable storage medium comprising instructions that, when executed by processing circuitry, cause the processing circuitry to:
for each of a plurality of periods, determine a respective value for each of a plurality of parameters of a patient with one or more medical devices;
for each of the plurality of periods, determine at least one of a heart failure risk score or status for the patient using one or more thresholds;
determine a number of the determinations for the patient that were false;
determine that that the number of false determinations for the patient satisfies a false determination criterion; and
modify at least one of the one or more thresholds for the patient in response to the determination that the number of false determinations for the patient satisfies the false determination criterion.

34: A system comprising:
one or more medical devices configured to, for each of a plurality of periods, determine a respective value for each of a plurality of parameters of a patient; and
processing circuitry configured to:
for each of the plurality of periods, execute an algorithm to determine at least one of a heart failure risk score or status for the patient based on the determined values for the period;
determine a number of the determined heart failure risk scores or statuses that were false determinations;
determine that the number of false determinations for the patient satisfies a false determination threshold; and
modify the algorithm for the patient in response to the determination that the number of false determinations for the patient satisfies the false determination threshold.

35: The system of claim 34, wherein the processing circuitry is configured to modify the algorithm by at least adjusting one or more thresholds.

36: The system of claim 35, wherein the processing circuitry is configured to execute the algorithm according to the parameters to:
determine an evidence level for each of the parameters based on the respective value for the parameter;
determine a risk level based on the plurality of evidence levels;
compare the risk level to a risk level threshold; and
determine the heart failure risk status based on the comparison of the risk level to the risk level threshold.

37: The system of claim 36, wherein the processing circuitry is configured to adjust the risk level threshold in response to the determination that the number of false determinations for the patient satisfies the false determination threshold.

38: The system of claim 37, wherein the false determination threshold comprises a false positive threshold, and the processing circuitry is configured to:
determine a number of the determinations for the patient that were false positives;
determine that the number of false positives satisfies the false positive threshold; and
increase the risk level threshold in response to the determination that the number of false positives satisfies the false positive threshold.

39: The system of claim 38, wherein the processing circuitry is configured to:
determine a value of the risk level threshold that makes at least one of the number of false positives a true negative; and
increase the risk level threshold based on the determined value.

40: The system of claim 36, wherein the processing circuitry is configured to execute the algorithm according to the parameters to:
apply a respective one or more evidence level thresholds to each of the plurality of parameter values; and
determine the evidence level for each of the parameters based on the comparison,
wherein the processing circuitry is further configured to adjust at least one of the evidence level thresholds in response to the determination that the number of false determinations for the patient satisfies the false determination threshold.

41: The system of claim 40, wherein the false determination threshold comprises a false negative threshold, and the processing circuitry is configured to:
determine a number of the determinations for the patient that were false negatives;
determine that the number of false negatives satisfies the false negative threshold; and
decrease the evidence level threshold in response to the determination that the number of false negatives satisfies the false negative threshold.

42: The system of claim 41, wherein the processing circuitry is configured to:
identify an evidence level threshold that was not satisfied for one of the number of false negatives and was satisfied for at least one prior true positive; and
decrease the identified evidence level threshold in response to the determination that the number of false negatives satisfies the false negative threshold.

43: The system of claim 34, wherein the false determination threshold comprises a threshold number of false determinations during a period of time.

44: The system of claim 34, wherein the processing circuitry is configured to determine the number of the determinations that were false based on at least one of user input or data from an electronic medical record system indicating whether the patient experienced a heart failure event.

45: A method comprising:
for each of a plurality of periods, determining a respective value for each of a plurality of parameters of a patient with one or more medical devices;
for each of the plurality of periods, executing an algorithm to determine at least one of a heart failure risk score or status for the patient based on the determined values for the period;
determining a number of the determined heart failure risk scores or statuses that were false determinations;
determining that the number of false determinations for the patient satisfies a false determination threshold; and
modifying the algorithm for the patient in response to the determination that the number of false determinations for the patient satisfies the false determination threshold.

46: The method of claim 45, wherein modifying the algorithm comprises adjusting one or more thresholds.

47: The method of claim 46, wherein executing the algorithm comprises:
determining an evidence level for each of the parameters based on the respective value for the parameter;
determining a risk level based on the plurality of evidence levels;
comparing the risk level to a risk level threshold; and
determining the heart failure risk status based on the comparison of the risk level to the risk level threshold.

48: The method of claim 47, wherein adjusting one or more thresholds comprises adjusting the risk level threshold in response to the determination that the number of false determinations for the patient satisfies the false determination threshold.

49-56. (canceled)

57: The method of claim 48, wherein the false determination threshold comprises a false positive threshold, and adjusting the risk level threshold comprises:
determining a number of the determinations for the patient that were false positives;
determining that the number of false positives satisfies the false positive threshold; and
increasing the risk level threshold in response to the determination that the number of false positives satisfies the false positive threshold.

58: The method of claim 49, wherein increasing the risk level threshold comprises:
determining a value of the risk level threshold that makes at least one of the number of false positives a true negative; and
increasing the risk level threshold based on the determined value.

59: The method of claim 47, wherein executing the algorithm comprises:
applying a respective one or more evidence level thresholds to each of the plurality of parameter values; and
determining the evidence level for each of the parameters based on the comparison,
wherein adjusting one or more thresholds comprises adjusting at least one of the evidence level thresholds in response to the determination that the number of false determinations for the patient satisfies the false determination threshold.

60: The method of claim 59, wherein the false determination threshold comprises a false negative threshold, and adjusting at least one of the evidence level thresholds comprises:
determining a number of the determinations for the patient that were false negatives;
determining that the number of false negatives satisfies the false negative threshold; and
decreasing the evidence level threshold in response to the determination that the number of false negatives satisfies the false negative threshold.

61: The method of claim 60, wherein decreasing the evidence level threshold comprises:
identifying an evidence level threshold that was not satisfied for one of the number of false negatives and was satisfied for at least one prior true positive; and
decreasing the identified evidence level threshold in response to the determination that the number of false negatives satisfies the false negative threshold.

62: The method of claim 45, wherein the false determination threshold comprises a threshold number of false determinations during a period of time.

63: The method of claim 45, wherein determining the number of determinations that were false comprises determining the number of the determinations that were false based on at least one of user input or data from an electronic medical record system indicating whether the patient experienced a heart failure event.
